SEQUENCE LISTING (23 sequences)

SEQ ID NO: 1 - RNA, 29903 nt, Severe acute respiratory syndrome coronavirus 2
  source 1..29903: mol_type = genomic RNA
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 2 - DNA, 24 bp, synthetic construct
  source 1..24: mol_type = other DNA
cctgggaaggtataaacctttaat

SEQ ID NO: 3 - DNA, 25 bp, synthetic construct
  source 1..25: mol_type = other DNA
tgttacctgggaaggtataaacctt

SEQ ID NO: 4 - DNA, 24 bp, synthetic construct
  source 1..24: mol_type = other DNA
ttttaaagttcgtttagagaacag

SEQ ID NO: 5 - DNA, 25 bp, synthetic construct
  source 1..25: mol_type = other DNA
aagttcgtttagagaacagatctac

SEQ ID NO: 6 - DNA, 24 bp, synthetic construct
  source 1..24: mol_type = other DNA
aggctctccatcttacctttcggt

SEQ ID NO: 7 - DNA, 25 bp, synthetic construct
  source 1..25: mol_type = other DNA
tacctgggaaggtataaacctttaa

SEQ ID NO: 8 - DNA, 25 bp, synthetic construct
  source 1..25: mol_type = other DNA
ttacctgggaaggtataaaccttta

SEQ ID NO: 9 - DNA, 25 bp, synthetic construct
  source 1..25: mol_type = other DNA
gttacctgggaaggtataaaccttt

SEQ ID NO: 10 - DNA, 25 bp, synthetic construct
  source 1..25: mol_type = other DNA
tgttacctgggaaggtataaacctt

SEQ ID NO: 11 - DNA, 25 bp, synthetic construct
  source 1..25: mol_type = other DNA
ttgttacctgggaaggtataaacct

SEQ ID NO: 12 - DNA, 25 bp, synthetic construct
  source 1..25: mol_type = other DNA
tttgttacctgggaaggtataaacc

SEQ ID NO: 13 - DNA, 25 bp, synthetic construct
  source 1..25: mol_type = other DNA
gtttgttacctgggaaggtataaac

SEQ ID NO: 14 - DNA, 25 bp, synthetic construct
  source 1..25: mol_type = other DNA
ggtttgttacctgggaaggtataaa

SEQ ID NO: 15 - DNA, 25 bp, synthetic construct
  source 1..25: mol_type = other DNA
tggtttgttacctgggaaggtataa

SEQ ID NO: 16 - DNA, 25 bp, synthetic construct
  source 1..25: mol_type = other DNA
ttggtttgttacctgggaaggtata

SEQ ID NO: 17 - DNA, 25 bp, synthetic construct
  source 1..25: mol_type = other DNA
gttggtttgttacctgggaaggtat

SEQ ID NO: 18 - DNA, 25 bp, synthetic construct
  source 1..25: mol_type = other DNA
ggttggtttgttacctgggaaggta

SEQ ID NO: 19 - DNA, 25 bp, synthetic construct
  source 1..25: mol_type = other DNA
tggttggtttgttacctgggaaggt

SEQ ID NO: 20 - DNA, 25 bp, synthetic construct
  source 1..25: mol_type = other DNA
cctcttacctcagttacaatttata

SEQ ID NO: 21 - protein, 14 aa, synthetic construct
  MOD_RES 2: note = 6-aminohexanoic acid
  MOD_RES 5: note = 6-aminohexanoic acid
  MOD_RES 8: note = 6-aminohexanoic acid
  MOD_RES 11: note = 6-aminohexanoic acid
  MOD_RES 13: note = 6-aminohexanoic acid
  MOD_RES 14: note = beta-alanine
RXRRXRRXRRXRXX

SEQ ID NO: 22 - DNA, 25 bp, synthetic construct
  source 1..25: mol_type = other DNA
ttgttacctgggaaggtataaacct

SEQ ID NO: 23 - DNA, 25 bp, synthetic construct
  source 1..25: mol_type = other DNA
gttcgtttagagaacagatctacaa

(19) United States
(12) Patent Application Publication
(10) Pub. No.: US 2023/0203492 A1
(43) Pub. Date: Jun. 29, 2023

(54) ANTISENSE THERAPEUTICS FOR BETACORONAVIRUS TREATMENT

(71) Applicants: Oregon State University, Corvallis, OR (US); The U.S.A., as Represented by the Secretary, Department of Health and Human Services, Bethesda, MD (US)

(72) Inventors: Hong M. Moulton, Corvallis, OR (US); David Adam Stein, Corvallis, OR (US); Heinrich Ulrich Feldmann, Hamilton, MT (US); Kyle Ture Rosenke, Hamilton, MT (US)

(73) Assignees: Oregon State University, Corvallis, OR (US); The U.S.A., as Represented by the Secretary, Department of Health and Human Services, Bethesda, MD (US)

(21) Appl. No.: 18/053,308

(22) Filed: Nov. 7, 2022

Related U.S. Application Data
(63) Continuation-in-part of application No. PCT/US21/31335, filed on May 7, 2021.
(60) Provisional application No. 63/021,859, filed on May 8, 2020.

(51) Int. Cl. C12N 15/113; A61P 31/14; A61P 11/00; A61K 47/54; A61K 47/64; A61K 31/7105

(52) U.S. Cl. CPC C12N 15/1131; A61P 31/14; A61P 11/00; A61K 47/549; A61K 47/64; A61K 31/7105; C12N 2310/11; C12N 2310/3513; C12N 2310/3125; C12N 2310/321; C12N 2310/3231; C12N 2310/3233

(57) ABSTRACT

Disclosed herein are embodiments of a compound useful for treating or preventing betacoronavirus infections such as SARS-Cov-2 infections. Also disclosed is a method for administering the compound to a subject, particularly a human subject, to treat or prevent a betacoronavirus infection in the subject. The compound can comprise an oligomer comprising a nucleic acid base sequence that is antisense to at least a portion of a SARS-CoV-2 genomic RNA, and can comprise a sequence present in the 5′ UTR and first 20 nt of coding sequence of the SARS-CoV-2 genomic RNA. The compound also can contain a peptide sequence. In some embodiments, the compound is a peptide-conjugated phosphorodiamidate morpholino oligomer (PPMO).

### CROSS-REFERENCES TO RELATED APPLICATIONS

This application is a continuation-in-part of PCT/US2021/031335, filed May 7, 2020, which claims the benefit of U.S. Provisional Application No. 63/021859, filed May 8, 2020, the disclosures of which are incorporated herein by reference in their entirety.

### STATEMENT REGARDING SEQUENCE LISTING

The Sequence Listing XML associated with this application is provided in XML format and is hereby incorporated by reference into the specification. The name of the XML file containing the sequence listing is 3014_P17US_Seq_List_20221030.xml. The XML file is 52 KB; was created on Oct. 30, 2022; and is being submitted electronically via Patent Center with the filing of the specification.

### FIELD

This disclosure concerns embodiments of compounds and methods useful for treating or preventing betacoronavirus infections, including embodiments of compounds for use in treating or preventing betacoronavirus infections.

### BACKGROUND

In December 2019, cases of an acute respiratory disease were reported from Wuhan, the capitol of Hubei province in China. The number of infections increased rapidly and spread to other areas of China and on Jan. 13, 2020, the first case was reported outside of China. The causative agent was identified as a novel coronavirus (CoV) of the lineage b of the genus Betacoronavirus that also includes the 2002 SARS-CoV that caused a global outbreak of severe acute respiratory syndrome (SARS) in 2002 and 2003. The newly emerged CoV was named SARS-CoV-2 by the World Health Organization (WHO) in February 2020, and the outbreak was declared as pandemic on Mar. 11, 2020. The respiratory disease caused by SARS-CoV-2 was named coronavirus 2019 disease (COVID-19). As of late Mar. 25, 2021, the WHO reports over 124 million cases and over 2.7 million deaths in 223 countries.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

Disclosed herein are embodiments of a compound, the compound can comprise an oligomer that can comprise a nucleic acid base sequence antisense to at least a portion of an RNA sequence of SARS-CoV-2, and a backbone comprising moieties that sterically block DNA and/or RNA cleavage. In some embodiments, the compound can further comprise a peptide. In some embodiments, the nucleic acid base sequence can be antisense to at least a portion of nucleotides 1-285 of the SARS-CoV-2 genomic RNA. In some embodiments, the nucleic acid base sequence can be antisense to at least a portion of nucleotides 1-50 of the SARS-CoV-2 genomic RNA. In some embodiments, the SARS-CoV-2 genomic RNA can have a sequence with at least 80% sequence identity to the sequence as set forth in SEQ ID NO: 1. In some embodiments, the oligomer can comprise a nucleic acid base sequence selected from SEQ ID NOs: 2-19, 22, and 23 or a nucleic acid base sequence having at least 90% sequence identity to one or more of SEQ ID NOs: 2-19, 22, and 23. In some embodiments, the oligomer can comprise a nucleic acid base sequence selected from SEQ ID NOs: 2-5, 22, and 23. In still other embodiments, the oligomer can comprise a nucleic acid base sequence selected from SEQ ID NO: 2, SEQ ID NO: 3, or SEQ ID NO: 22. In some embodiments, the oligomer backbone can comprise phosphorodiamidate morpholino (PMO), methylphosphonate, 2′-O-methyl RNA (2′-)Me), 2′-O-methyl phosphorothioate (2′-OMePS), 2′-O-methoxyethyl RNA (2′-MOE), 2′-O-methoxyethyl phosphorothioate (2′-MOE-PS), peptide nucleic acid (PNA), tricycle-DNA (tcDNA), locked nucleic acid (LNA), or a combination thereof. In some embodiments, the oligomer backbone can comprise a structure selected from

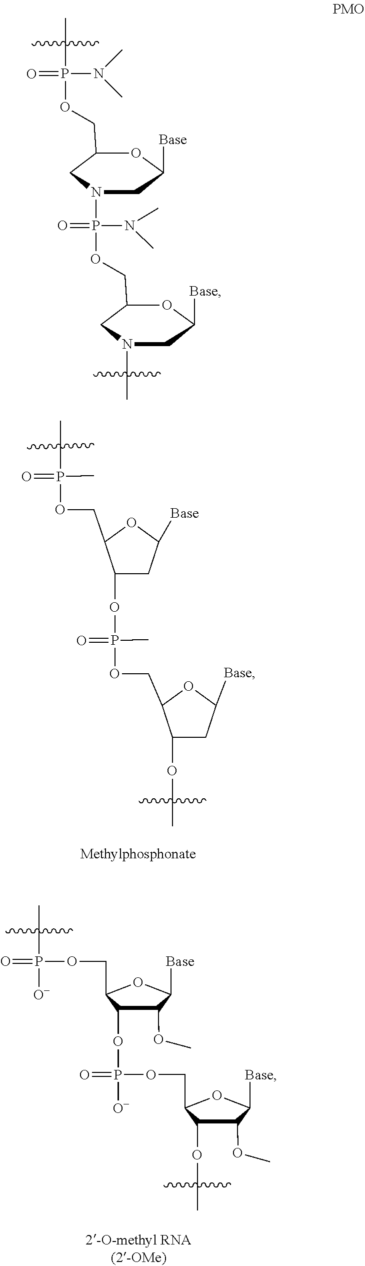

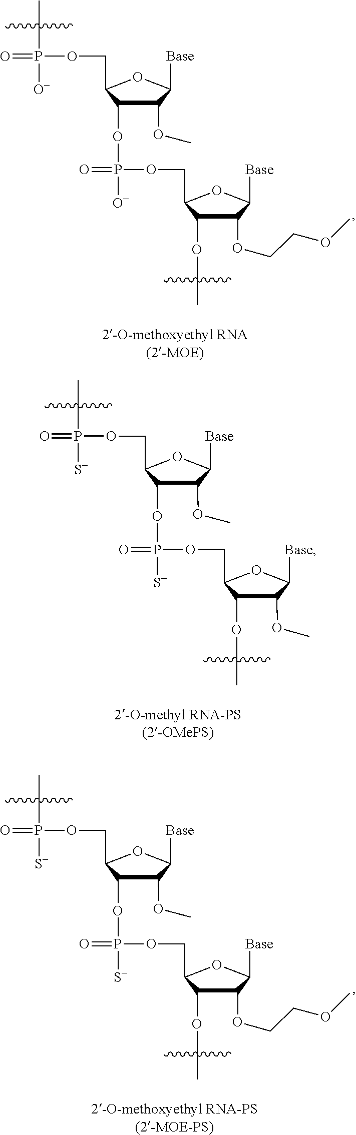

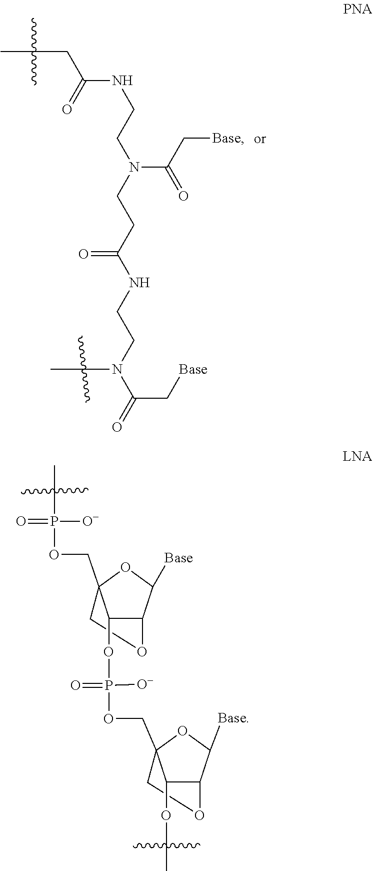

still other embodiments, the peptide can have a length from 2 to 60 amino acids. In still other embodiments, the peptide can comprise one or more amino acids selected from glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, proline, beta-alanine, selenocysteine, pyrrolysine, 7-aminoheptanoic acid, 6-amino hexanoic acid, 5-aminopentanoic acid, 4-aminobutanoic acid, homoarginine, or amino acids containing a poly(oxyethylene) group. In still other embodiments, the peptide can comprise a sequence as set forth in SEQ ID NO: 21, or wherein the peptide has a sequence with at least 90% sequence identity to the sequence as set forth in SEQ ID NO: 21. In some embodiments, the peptide can be attached at the 3′ end of the oligomer, wherein the peptide is attached directly to the oligomer backbone or indirectly to the oligomer backbone through a linker. In still other embodiments, the peptide can be attached at the 5′ end of the oligomer, wherein the peptide is attached directly to the oligomer backbone or indirectly to the oligomer backbone through a linker. In some embodiments, the compound can have a structure according to Formula 1

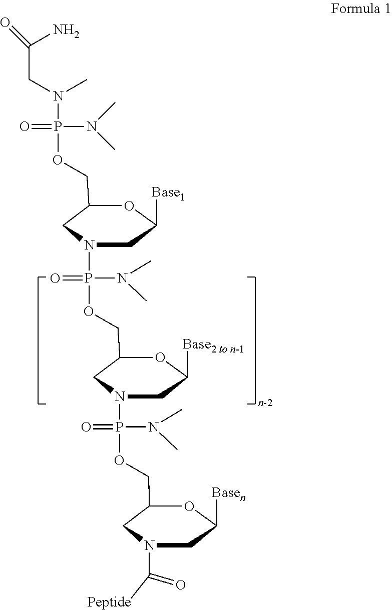

wherein: n is from 2 to 50; each base independently is selected from adenine, guanine, cytosine, thymine or uracil; and peptide is a peptide comprising from 2 amino acid to 60 amino acids. In some embodiments, the compound can have a structure according to Formula 2

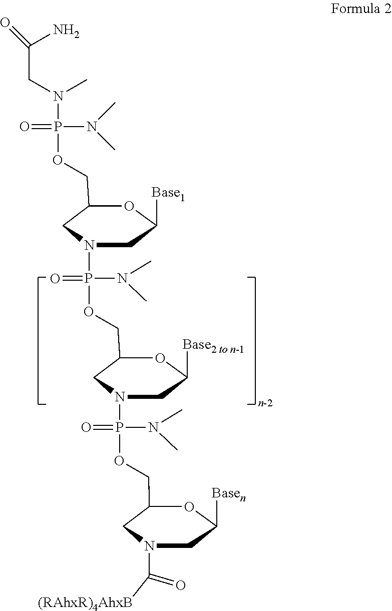

wherein R is Arginine, Ahx is 6-aminohexanoic acid, and B is beta-alanine. In still other embodiments, Base₁ to Baseₙ in Formula 2 can be SEQ ID NO: 2, SEQ ID NO: 3, or SEQ ID NO: 22.

In another aspect, disclosed herein are embodiments of a method of treating or preventing a SARS-CoV-2 infection, comprising administering to a subject a compound as described above.

In another aspect, disclosed herein are embodiments of a method for treating or preventing a SARS-CoV-2 infection in a human subject, comprising administering to the subject an effective amount of a compound having a structure

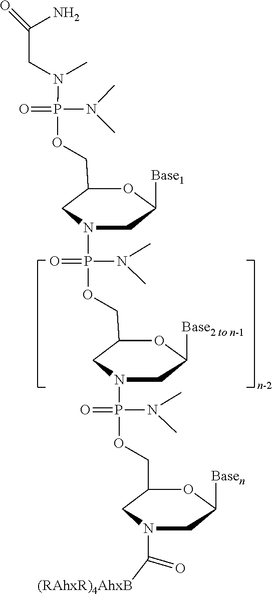

or a pharmaceutically acceptable salt thereof, wherein: n is from 20 to 30; each Base independently is selected from adenine, guanine, cytosine or thymine; R is Arginine; Ahx is 6-aminohexanoic acid; and B is beta-alanine.

The foregoing and other objects, features, and advantages of the invention will become more apparent from the following detailed description, which proceeds with reference to the accompanying figures.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 provides an exemplary general formula for compounds suitable for use in the disclosed method and illustrates the structural features of a peptide phosphorodiamidate morpholino oligomers (PPMO).

FIG. 2 is a graph of TCID50 (virus titrations) versus time post-infection, illustrating the effect on SARS-CoV-2 replication of an exemplary 5′End 1 (SEQ ID NO: 2) PPMO at different concentrations.

FIG. 3 is a graph of TCID50 versus time post-infection, illustrating the effect on SARS-CoV-2 replication of an exemplary 5′End 2 (SEQ ID NO: 3) PPMO at different concentrations.

FIG. 4 is a graph of TCID50 versus time post-infection, illustrating the effect on SARS-CoV-2 replication of an exemplary TRS 1 (SEQ ID NO: 4) PPMO at different concentrations.

FIG. 5 is a graph of TCID50 versus time post-infection, illustrating the effect on SARS-CoV-2 replication of an exemplary TRS 2 (SEQ ID NO: 5) PPMO at different concentrations.

FIG. 6 is a graph of TCID50 versus time post-infection, illustrating the effect on SARS-CoV-2 replication of an exemplary AUG (SEQ ID NO: 6) PPMO at different concentrations.

FIG. 7 is a graph of TCID50 versus time post-infection, illustrating the effect on SARS-CoV-2 replication of the negative control PPMO comprising a control sequence (SEQ ID NO: 20) at different concentrations.

FIG. 8 is a graph of quantitative reverse transcription polymerase chain reaction (qRT-PCR) versus time post infection, illustrating the effect on SARS-CoV-2 replication of an exemplary 5′End 1 (SEQ ID NO: 2) PPMO at various concentrations.

FIG. 9 is a graph of quantitative reverse transcription polymerase chain reaction (qRT-PCR) versus time post infection, illustrating the effect on SARS-CoV-2 replication of an exemplary 5′End 2 (SEQ ID NO: 3) PPMO at various concentrations.

FIG. 10 is a graph of quantitative reverse transcription polymerase chain reaction (qRT-PCR) versus time post infection, illustrating the effect on SARS-CoV-2 replication of an exemplary TRS 1 (SEQ ID NO: 4) PPMO at various concentrations.

FIG. 11 is a graph of quantitative reverse transcription polymerase chain reaction (qRT-PCR) versus time post infection, illustrating the effect on SARS-CoV-2 replication of an exemplary TRS 2 (SEQ ID NO: 5) PPMO at various concentrations.

FIG. 12 is a graph of quantitative reverse transcription polymerase chain reaction (qRT-PCR) versus time post infection, illustrating the effect on SARS-CoV-2 replication of an exemplary AUG (SEQ ID NO: 6) PPMO at various concentrations.

FIG. 13 is a graph of quantitative reverse transcription polymerase chain reaction (qRT-PCR) versus time post infection, illustrating the effect on SARS-CoV-2 replication of the negative control PPMO comprising a control sequence (SEQ ID NO: 20) at various concentrations.

FIG. 14 provides structures of exemplary oligomer backbone structures with steric-blocking moieties that resist cleavage when administered to a subject.

FIG. 15 provides alternative PMO structures suitable for use in the disclosed compounds.

FIG. 16 is a graph of in vivo efficacy of PPMO versus SARS-CoV-2 in a mouse model. Specifically, this graph provides plaque assay data that illustrates the PPMO 5′END-2 (SEQ ID NO: 3) (which is designed to target SARS-CoV-2 nt 5-29) was able to suppress viral titer by approximately 80-90%. The PPMO TRS-1 was not statistically different from virus infection control (PBS) or NC705 (negative control PPMO).

### DETAILED DESCRIPTION

While illustrative embodiments have been illustrated and described, it will be appreciated that various changes can be made therein without departing from the spirit and scope of the invention.

I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified. All sequences associated with the GenBank Accession NOs. mentioned herein are incorporated by reference in their entirety as of the present application's priority date.

As used herein, the term “oligomer” is a low molecular weight molecule consisting of a small plurality of units, wherein the smally plurality of units can include, but are not limited to, nucleotides.

By “antisense” is meant a nucleic acid sequence that is the reverse complement to a second specific nucleic acid sequence.

The phrase “steric-blocking antisense oligomers” refers to a mechanism of action where the oligomer binds to a complementary RNA sequence and physically prevents or inhibits the translational machinery required for gene expression.

“Backbone” refers to the structural framework of nucleic acids. The backbone can include bonds and/or structural moieties that are resistant to degradation from cellular DNA and/or RNA cleavage mechanisms.

The term “sequence homology” refers to resemblance (i.e., similarity) between two sequences. The sequences can be nucleotide sequences or amino acids sequences. Sequence alignment tools such as BLAST, or any other tools used by those of ordinary skill in the art, can be used to assess sequence homology (e.g., BLASTN for nucleotide sequences and BLASTP for amino acid sequences).

The term “sequence identity” refers to the occurrence of exactly the same nucleotide or amino acid in the same position following a sequence alignment to a reference sequence.

The term “peptide” means a compound comprising two or more amino acids linked in a chain.

The term “linker” as used herein, refers to any of the well-known cleavable or non-cleavable linkers that can be used to conjugate a PPMO to a cell-penetrating peptide. Methods of conjugating a PPMO to a cell-penetrating peptide through a cleavable or non-cleavable linker can be any of the methods well-known to one of ordinary skill in the art.

As used herein, “treat,” “treating,” “treatment,” “prevent,” or “preventing” refer to both therapeutic treatment or prophylactic measures. Prophylactic measures prevent a subject from being infected by the SARS-CoV-2 virus. Therapeutic treatment results in the amelioration or eradication of a SARS-CoV-2 infection and/or an improvement, such as an easing or ceasing, of one or more symptoms associated with a SARS-CoV-2 infection, such that the subject experiences and/or reports an improvement in feeling or condition, even if the subject is still infected with the SARS-CoV-2 virus. Therapeutic treatment can also include halting or slowing the progression of disease caused by SARS-CoV-2, regardless of whether improvement is realized.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

When chemical structures are depicted or described, unless explicitly stated otherwise, all carbons are assumed to include implicit hydrogens such that each carbon conforms to a valence of four. For example, in the structure on the left-hand side of the schematic below there are nine hydrogen atoms implied. The nine hydrogen atoms are depicted in the right-hand structure.

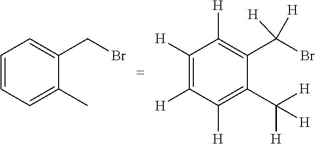

Sometimes a particular atom in a structure is described in textual formula as having a hydrogen or hydrogen atoms, for example -CH2CH2-. It will be understood by a person of ordinary skill in the art that the aforementioned descriptive techniques are common in the chemical arts to provide brevity and simplicity to description of organic structures.

SARS-CoV-2 genomic RNA: The genomic RNA sequence of a SARS-CoV-2 virus. An exemplary SARS-CoV-2 genomic RNA sequence is provided by SEQ ID NO: 1. However, a person of ordinary skill in the art understands that the term SARS-CoV-2 genomic RNA can refer to any SARS-CoV-2 genomic RNA sequence, such as a SARS-CoV-2 RNA sequence having at least 90% sequence identity (for example, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO:1, such as at least 95% (for example, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 1. Additionally, multiple examples of SARS-CoV-2 genomic RNA sequences have been identified and are suitable for use in the present disclosure, and such nucleic acid sequences are publicly available. For example, GenBank Accession NOs. MT007544.1, MT114419.1, MT077125.1, MT374102.1, MT415321.1, MT359865.1, MT371570.1, MT370954.1, MT419820.1, and MT412307.1, all of which are incorporated herein by reference as present in GenBank as of the present application's priority date.

Sequence identity/similarity: The identity/similarity between two or more nucleic acid sequences, or between two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. In some embodiments, one or more disclosed peptides can comprise one or more amino acid sequences having at least 90% sequence identity (for example, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 21, such as at least 95% (for example, 95%, 96%, 97%, 98%, 99%, 99.1%, 99.2%, 99.3%, 99.4%, 99.5%, 99.6%, 99.7%, 99.8%, 99.9%, or 100%) to SEQ ID NO: 21. In some embodiments, a disclosed compound can comprise an oligomer comprising a nucleic acid base sequence according to SEQ ID NOs: 2-19 and 22-24 or the compound can comprise an oligomer comprising a nucleic acid base sequence having at least 90% sequence identity (for example, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, or 100%) to one or more of SEQ ID NOs: 2-19 and 22-24 such as at least 95% (for example, 95%, 96%, 97%, 98%, 99%, 99.1%, 99.2%, 99.3%, 99.4%, 99.5%, 99.6%, 99.7%, 99.8%, 99.9%, or 100%) sequence identity to one or more of SEQ ID NOs: 2-19 and 22-24.

Sequence alignment methods for comparison and to determine sequence identity or similarity are known to those of ordinary skill in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the internet, for use in connection with the sequence analysis programs blastp, blastn, blastx, tblastn and tblastx. Additional information can be found at the NCBI web site.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

II. Overview

Coronaviruses (CoV) are a large group of enveloped, single-stranded positive-sense RNA viruses belonging to the order Nidovirales that infect a broad range of mammalian and avian species, typically causing respiratory and/or enteric tract disease. Betacoronaviruses are a subgenus of coronaviruses that include SARS-CoV-2, as well as SARS and MERS (Middle East respiratory syndrome virus). The 5′UTR of the coronavirus genome contains sequences and structures known to be important in various aspects of the virus life-cycle including translation and RNA synthesis. In an initial study designed to test the ability of PPMO to act as antiviral inhibitors of SARS-CoV-2 replication, seven PPMO (e.g., SEQ ID NOs: 1-5, 22, and 23) were designed to target various 24-25 nucleotide sites within the 5′UTR and first 11 nucleotides of the coding sequence for SARS-CoV-2 positive sense genomic RNA.

As with other positive strand viruses that utilize cap-dependent translation, access of trans-acting proteins to the 5′-terminal region of the nidoviral genome is critical to the process of translation pre-initiation. Three of the exemplary PPMO in this study target the 5′-terminal-region of the genome. The 5′END-1 PPMO targets the 5′ terminal nucleotides 1-24 of the SARS-CoV-2 genome, 5′END-2 targets nucleotides 5-29 in the 5′UTR, and 5′END-3 targets nucleotides 6-30 in the 5′UTR. 5′END-1, 5′END-2, and 5′END-3 were designed with the intention of interfering with the pre-initiation of the translation of the genomic and various subgenomic mRNAs. Regarding 5′END-1 and 5′END-2, 5′END-1 has a lower predicted thermal melting temperature with its target than does 5′END-2 (78° C. and 87° C., respectively). However, 5′END-1 obstructs the first few nucleotides in the terminus of the positive-sense viral genome which can be of particular importance for assembly of the translation pre-initiation complex and/or capping of nascent viral mRNAs. It can be inferred from previous RNA structure modeling of SARS-CoV that the first 6 nucleotide of the SARS-CoV-2 genome are not part of Stem-Loop 1 (SL1). Mfold analysis (data not shown) also indicates the presence of a stem-loop formation from nucleotides 7-34 of the SARS-CoV-2 genome.

It is generally accepted that coronaviruses use the process of discontinuous subgenomic mRNA synthesis to produce mRNAs. In this process, full-length genomic minus strand RNAs as well as a 5′ nested set of subgenomic minus strand RNAs are first synthesized from genomic RNA and serve as templates for genomic and subgenomic mRNA synthesis. The transcription regulatory sequence (TRS) is a six-nine nucleotide sequence that is implicated in the production of negative strand mRNA templates during discontinuous mRNA synthesis. Three PPMO were designed to target the TRS region in the 5′UTR and thereby potentially interfere with body-TRS to leader-TRS base-pairing. The leader-TRS-region targeted PPMO also have the potential to interfere with the process of translation, by blocking translocation of the 48S translation preinitiation complex along the 5′UTR of various viral mRNAs. Both TRS-directed PPMO were designed to target the SARS-CoV-2 leader-TRS (5′-ACGAAC-3′), with TRS-1 also targeting at least 7 nucleotides on each side of the leader-TRS core-sequence. TRS-2 and TRS-3 target the leader-TRS along with 17 nucleotides to the viral 5′ side, and therefore a contiguous 23 of its 25 residues are complementary to sequence likely present on both genomic and several of the sub-genomic mRNAs.

The TRS-leader is important in subgenomic mRNA synthesis and is located at nt 70-75. Based on previous studies on coronaviruses and other nidoviruses, all of the SARS-CoV-2 subgenomic mRNAs likely include the first 75 bases of genomic RNA sequence, but are unlikely to include sequence 3′ from base 75 of the 5′ UTR. The TRS-3 PPMO is designed to target bases 51-75, to improve the likelihood of binding to all subgenomic RNAs, as well as genomic RNA. By binding to the various subgenomic RNAs in their respective 5′ UTRs, at the nucleotides represented by nt 51-75 of the genomic RNA, the TRS-3 PPMO can be expected to interfere with the preintiation of translation of some or all of the subgenomic mRNAs, as well as potentially interfering with the body-to-leader-TRS base pairing during subgenomic mRNA transcription of all subgenomic mRNAs, as described above.

The AUG PPMO spans the AUG translation initiation codon region for ORF1a/b, which codes for the viral replicase polyprotein, and was designed to block the initiation of translation. The translation start site region has been a typical and productive target for PMO-technology in general, especially in cellular genes.

III. Compounds

Disclosed herein are embodiments of steric-blocking antisense oligomers useful for treating and/or preventing SARS-CoV-2 infections. Also disclosed herein are embodiments of steric-blocking antisense oligomers for use as a medicament. In some embodiments, are described the steric-blocking oligomers for use in treating or preventing SARS-CoV-2 infections. In some embodiments, are described the steric-blocking oligomers for use in treating or preventing SARS-CoV-2 infections in humans. In still other embodiments, the steric-blocking oligomer can further be conjugated to a peptide for the purpose of cellular delivery and/or tissue targeting.

In some embodiments, the compound can comprise one or more oligomers that comprise a nucleic acid base sequence that is antisense to at least a portion of the RNA sequence of SARS-CoV-2. In other embodiments, the compound can comprise one or more oligomers that comprise a nucleic acid base sequence that is antisense to at least a portion of the RNA sequence of SARS-CoV-2, wherein the oligomer is conjugated to a peptide for the purpose of cellular delivery and/or tissue targeting as further described below.

In some embodiments, the compound can comprise one or more oligomers that comprise a nucleic acid base sequence that is antisense to at least a portion of the RNA sequence of SARS-CoV-2. The oligomer's nucleic acid base sequence can comprise, consist essentially of, or consist of from 2 to 50 or more bases, from 5 to 50 bases, from 10 to 40 bases, from 10 to 30 bases, from 15 to 30 bases, or from 20 to 30 bases, and in some embodiments, the oligomer comprises a sequence of 24 bases or 25 bases.

In some embodiments, the compound comprises an oligomer that comprise a nucleic acid base sequence that is antisense to an RNA sequence located in nucleotides 1-300 of the SARS-CoV-2 genome. The RNA sequence can be an RNA sequence located in the SARS-CoV-2 5′UTR and/or first 20 nucleotides of the coding sequence, that is, the RNA sequence can be located in nucleotides 1-285 of the SARS-CoV-2 genomic RNA.

In some embodiments, the compound comprises an oligomer that comprises a nucleic acid base sequence that is antisense to at least a portion of the 5′ terminal region of a SARS-CoV-2 genomic RNA sequence, such as antisense to at least a portion of nucleotides 1-50, nucleotides 1-40, or nucleotides 1-30 of a SARS-CoV-2 genomic RNA. In certain embodiments, the oligomer comprises a nucleic acid base sequence that is antisense to nucleotides 1-24, nucleotides 5-29, or nucleotides 6-30 of a SARS-CoV-2 genomic RNA, and/or can have a sequence according to SEQ ID NOs: 2, 3, or 22 (Table 1).

In some embodiments, the compound comprises an oligomer that comprises a nucleic acid base sequence that is antisense to at least a portion of the TRS-leader sequence, such as antisense to at least a portion of nucleotides 50-90, nucleotides 50-85, or nucleotides 53-82 of the SARS-CoV-2 genomic RNA. In certain embodiments, the oligomer comprises a nucleic acid base sequence that is antisense to nucleotides 51-75, 53-77, or nucleotides 59-82 of a SARS-CoV-2 genomic RNA, and/or can have a sequence according to SEQ ID NOs: 4, 5, or 23 (Table 1).

In some embodiments, the compound comprises an oligomer that comprises a nucleic acid base sequence that is antisense to at least a portion of the AUG translation start site region, such as antisense to at least a portion of nucleotides 245-285, or nucleotides 251-275 of a SARS-CoV-2 genomic RNA, for example, SEQ ID NO: 6 (Table 1).

Tables 1 and 2 provide exemplary nucleic acid base sequences suitable for use in the disclosed compounds. Table 1 also provides possible target regions in a SARS-CoV-2 genomic RNA based on GenBank Accession No. NC045512 (SEQ ID NO:1).

TABLE 1
Exemplary sequences suitable for use in the
disclosed compounds

| | | Target | |
|---|---|---|---|
| | | location in | SEQ |
| PPMO | | a SARS-2 | ID |
| Name | PMO sequence | genome* | NO: |
| 5′END-1 | CCTGGGAAGGTATAAACCTTTAAT | 1-24 | 2 |
| 5′END-2 | TGTTACCTGGGAAGGTATAAACCTT | 5-29 | 3 |
| 5′END-3 | TTGTTACCTGGGAAGGTATAAACCT | 6-30 | 22 |
| TRS-1 | TTTTAAAGTTCGTTTAGAGAACAG | 59-82 | 4 |
| TRS-2 | AAGTTCGTTTAGAGAACAGATCTAC | 53-77 | 5 |
| TRS-3 | GTTCGTTTAGAGAACAGATCTACAA | 51-77 | 23 |
| AUG-1 | AGGCTCTCCATCTTACCTTTCGGT | 251-275 | 6 |
| Negative | CCTCTTACCTCAGTTACAATTTATA | N/A | 20 |
| Control | | | |

*based on GenBank Accession # NC045512

TABLE 2
Additional nucleic acid base sequences
suitable for targeting the 5′ terminal
region of a SARS-CoV-2 genomic RNA

| SEQ ID NO: | PMO Sequences |
|---|---|
| 7 | TACCTGGGAAGGTATAAACCTTTAA |
| 8 | TTACCTGGGAAGGTATAAACCTTTA |
| 9 | GTTACCTGGGAAGGTATAAACCTTT |
| 10 | TGTTACCTGGGAAGGTATAAACCTT |
| 11 | TTGTTACCTGGGAAGGTATAAACCT |
| 12 | TTTGTTACCTGGGAAGGTATAAACC |
| 13 | GTTTGTTACCTGGGAAGGTATAAAC |
| 14 | GGTTTGTTACCTGGGAAGGTATAAA |
| 15 | TGGTTTGTTACCTGGGAAGGTATAA |
| 16 | TTGGTTTGTTACCTGGGAAGGTATA |
| 17 | GTTGGTTTGTTACCTGGGAAGGTAT |
| 18 | GGTTGGTTTGTTACCTGGGAAGGTA |
| 19 | TGGTTGGTTTGTTACCTGGGAAGGT |

Regarding the PPMO target sites directed to SARS-CoV-2 and whether these target sites would change in a SARS-CoV-2 variant, the PPMO target sites are highly conserved in SARS-CoV-2 variants. The virus-targeted PPMO in this study were designed based on the SARS-CoV-2 GenBank Reference Sequence (NC_045512). As of this writing, there are no reported mutations at the 5′END-2, 5′END-3, TRS-1, TRS-2, or TRS-3 PPMO target sites in available reference sequences for the SARS-CoV-2 lineages of Alpha (B.1.1.7), Beta (B.1.351), Gamma (P.1), Delta (B1.617.2), or Omicron variants (BA.2, BA.4, BA.5).

However, even if a single mutation at a PPMO target site were to evolve, the PPMO would still work well, as previous studies have shown that PPMOs having a single base mismatch with their target site retain approximately 90% of their activity compared to those having perfect agreement, suggesting that minor sequence divergence at PPMO target sites will not substantially reduce antiviral activity.

The oligomer(s) can further comprise a backbone that comprises bonds and/or structural moieties that are resistant to degradation when administered to a subject and/or exposed to typical cellular DNA and/or RNA cleavage mechanisms, such as mechanisms suitable to cleave the phosphate linkages in DNA or RNA. In some embodiments, moieties on the backbone sterically block DNA and/or RNA cleavage mechanisms. Suitable backbones include, but are not limited to, phosphorodiamidate morpholino (PMO), methylphosphonate, 2′-O-methyl RNA (2′-OMe), 2′-O-methyl phosphorothioate (2′-OMePS), 2′-O-methoxyethyl RNA (2′-MOE), 2′-O-methoxyethyl phosphorothioate (2′-MOE-PS), peptide nucleic acid (PNA), tricycle-DNA (tcDNA), locked nucleic acid (LNA), or a combination thereof. Exemplary backbone moieties are illustrated below:

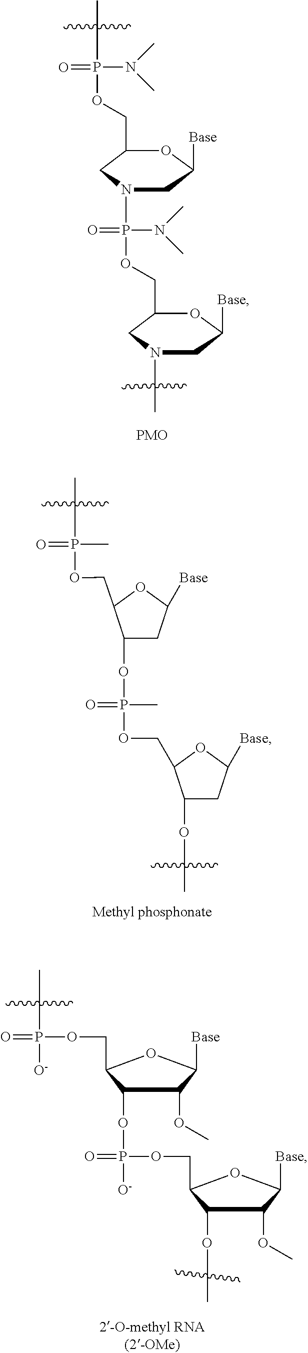

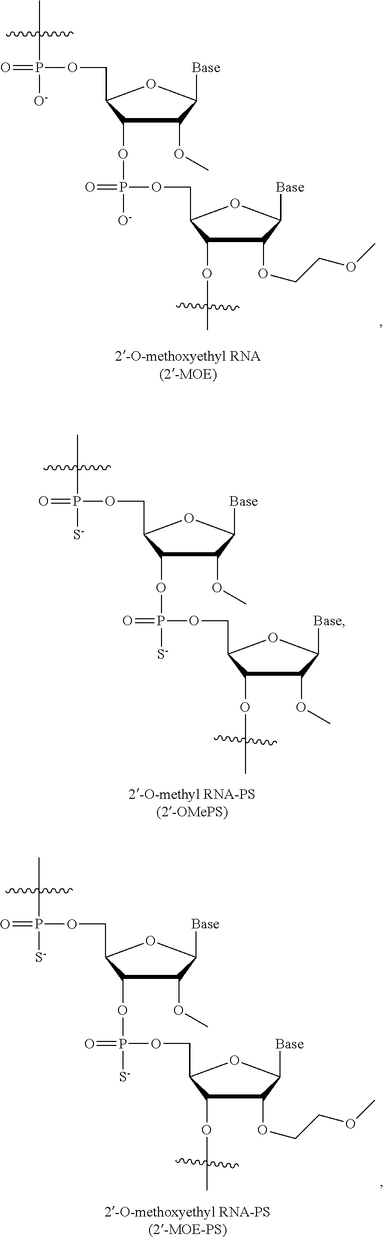

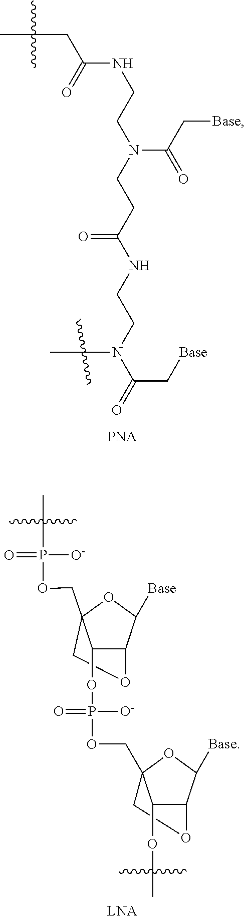

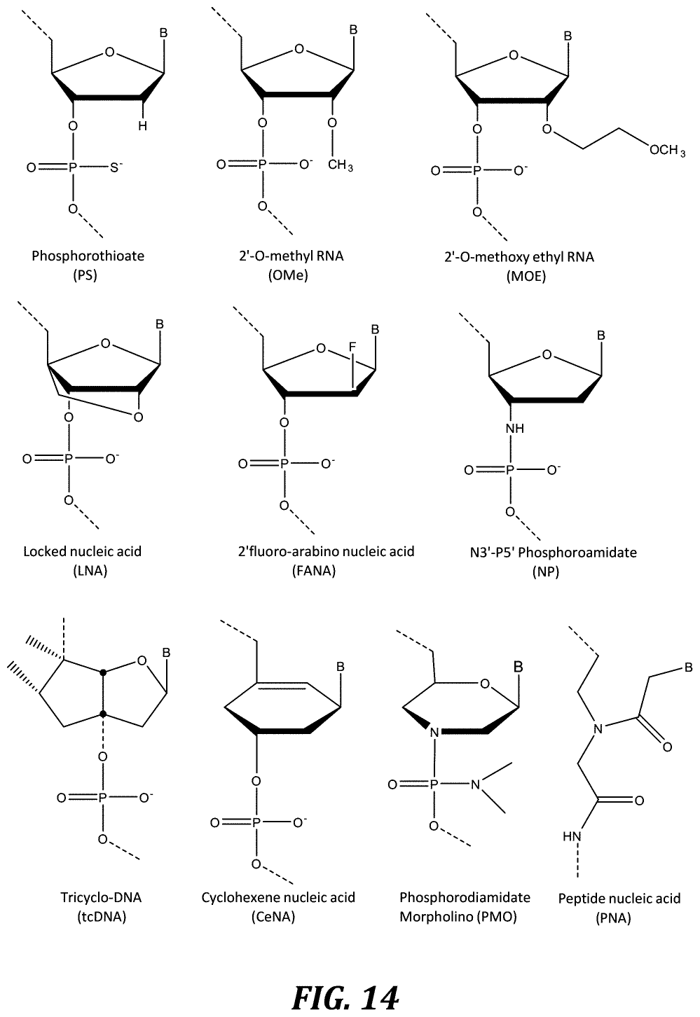

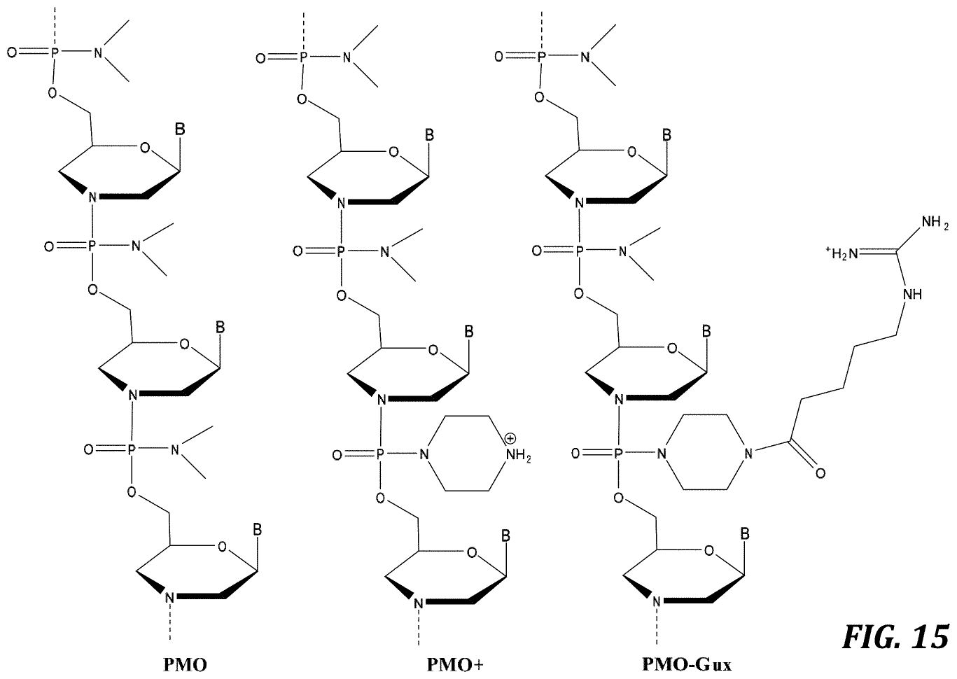

FIGS. 14 and 15 provide additional exemplary backbone moieties suitable for use in the disclosed compounds. FIG. 14 provides exemplary monomer units suitable for use in the backbone structure of the disclosed compound. And FIG. 15 provides examples of modified PMO structures, such as charged structures comprising one or more piperazine moieties that optionally can be substituted, such as with an amino acid. A person of ordinary skill in the art understands that the nucleic acid backbone of the disclosed compound can comprise, consist essentially of, or consist of, one of the monomer unit types disclosed herein, or it can comprise, consist essentially of, or consist of, more than one type of monomer unit, such as 2, 3, 4, 5, 6, or more monomer unit types.

Certain exemplary nucleic acid base sequences suitable for use in the disclosed compounds are provided in Tables 1 and 2. A person of ordinary skill in the art understands that with respect to the nucleic acid sequences disclosed herein, A, G, C, T, and U represent bases adenine, guanine, cytosine, thymine and uracil, respectively, as shown below, where the wavy line indicates the point of attachment to the oligomer backbone.

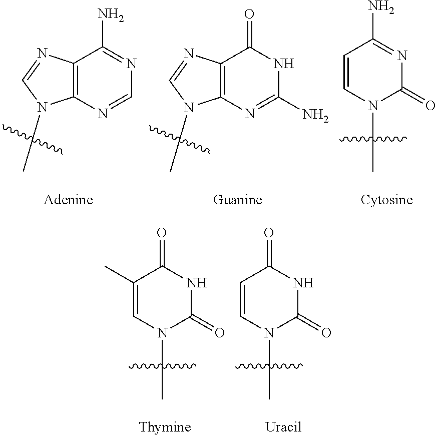

In some embodiments, the compound further comprises a peptide sequence covalently attached to the oligomer, and the compound can have a formula: Peptide-Oligomer, Peptide-Oligomer-Peptide, Peptidel-Oligomer-Peptide2, or Peptidel-Peptide2-Oligomer, where Peptidel and Peptide2 have different amino acid sequences. Additionally, a peptide can be in either linear or branched form. In other embodiments, the compound comprises a peptide sequence.

The peptide can be of any length suitable to facilitate transport of the compound. In some embodiments, the peptide comprises, consists essentially of, or consists of, from 2 amino acids to 60 amino acids or more, such as from 2 amino acids to 40 amino acids, from 5 to 30 amino acids, from 5 to 20 amino acids, from 10 to 20 amino acids or from 10 to 15 amino acids. In certain disclosed embodiments, the peptide has a length of 14 amino acids.

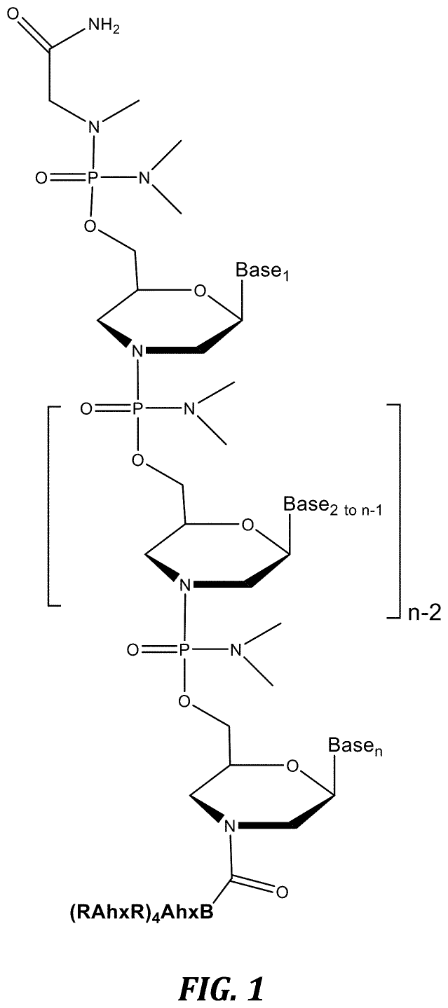

In certain embodiments, the oligomer comprises a PMO backbone, and the compound can be a peptide-conjugated PMO (PPMO). The peptide can be selected and/or designed to facilitate transport of the compound, such as through a membrane and/or into a cell. The peptide can be a naturally occurring sequence, such as a protein or fragment thereof, or the peptide can be a non-naturally occurring amino acid sequence. FIG. 1 provides an exemplary chemical structure of a PPMO. With respect to the example in FIG. 1, n is the number of nucleic acid bases in the compound, R is arginine, Ahx is 6-aminohexanoic acid, B is beta-alanine, and each Base indicates a nucleic acid base. For example, with respect to FIG. 1, when n is 24 and Base₁ to Base₂₄ is CCTGGGAAGGTATAAACCTTTAAT (SEQ ID NO: 2), the nucleic acid sequence corresponds to 5′END-1, and when n is 25 and Base₁ to Base₂₅ is TGTTACCTGGGAAGGTATAAACCTT (SEQ ID NO: 3) the nucleic acid sequence corresponds to 5′END-2 or TTGTTACCTGGGAAGGTATAAACCT (SEQ ID NO: 22), the nucleic acid sequence corresponds to 5′END-3.

However, a person of ordinary skill in the art understands that the suitable peptides can comprise any amino acid, such as one or more of natural amino acids, such as glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, or proline, and such amino acids can be the L-amino acid, the D-amino acid or a mixture thereof. In some embodiments, a natural amino acid in the peptide is the L-amino acid. Additionally, or alternatively, the peptide can comprise one or more alternative naturally occurring or non-naturally occurring amino acids, for example, beta-alanine, selenocysteine, pyrrolysine, 7-aminoheptanoic acid, 6-amino hexanoic acid, 5-aminopentanoic acid, 4-aminobutanoic acid, homoarginine, or amino acids containing a poly(oxyethylene) group.

The peptide can be attached to the oligomer via the oligomer backbone and can be attached at the 3′ end of the oligomer, such as in FIG. 1, or it can be attached to the 5′ end of the oligomer. Additionally, the peptide can be attached to the oligomer by any suitable bond, such as an amide bond (as shown in FIG. 1), maleimide bond, a disulfide bond, an ester bond, or a bond formed by “click” chemistry with or without being catalyzed by copper ions. In still other embodiments, the peptide can be attached to the oligomer by any suitable linker, wherein the linker can be any suitable cleavable linker or any suitable non-cleavable linker. In still other embodiments, the peptide can be attached at the 3′ end of the oligomer through any suitable cleavable linker or any suitable non-cleavable linker. In still other embodiments, the peptide can be attached at the 5′ end of the oligomer through any suitable cleavable linker or any suitable non-cleavable linker.

Exemplary peptides useful in the disclosed technology include, but are not limited to, the exemplary protein sequence provided by SEQ ID NO: 21. In particular embodiments, the peptide is RAhxRRAhxRRAhxRRAhxRAhxB where R=Arginine, Ahx=6-aminohexanoic acid, and B =beta-alanine (SEQ ID NO: 21).

In some embodiments, the compound has a structure according to Formula 1

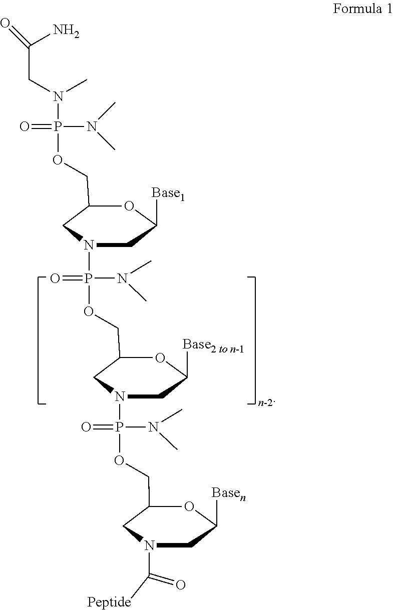

With respect to Formula 1, n is from 2 to 50, such as from 5 to 50, from 10 to 40, from 15 to 30 or from 20 to 30, and in certain embodiments, n is 24 and in other particular embodiments, n is 25. Each base independently is selected from adenine, guanine, cytosine, thymine, or uracil, and can be selected from adenine, guanine, cytosine, or thymine. And Peptide is a peptide as disclosed herein. In some embodiments, the peptide is SEQ ID NO: 21.

In particular embodiments, the compound can have a structure according to Formula 2

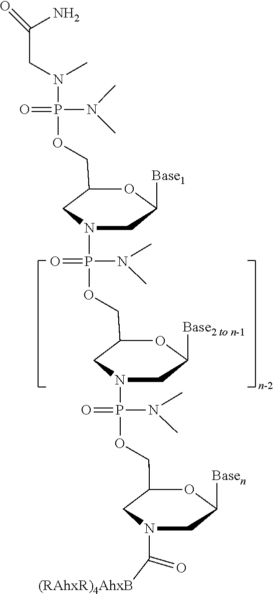

With respect to Formula 2, n and each base are as defined for Formula 1. R is Arginine, Ahx is 6-aminohexanoic acid, and B is beta-alanine.

In particular exemplary embodiments of Formula 2, n is 24 and Base₁ to Base₂₄ is CCTGGGAAGGTATAAACCTTTAAT (SEQ ID NO: 2), or n is 25 and Base₁ to Base₂₅ is TGTTACCTGGGAAGGTATAAACCTT (SEQ ID NO: 3) or TTGTTACCTGGGAAGGTATAAACCT (SEQ ID NO: 22).

IV. Method for Administering the Compounds

A. Formulation and Administration

The disclosed compounds described herein are described for use as a medicament. In some embodiments, the disclosed compound(s) are described for use in treating or preventing a SARS-CoV-2 infection. In other embodiments, the disclosed compound(s) are described for use in treating or preventing a SARS-CoV-2 infection in a human.

The disclosed compounds can be formulated as pharmaceutical compositions and administered to a mammalian host, such as a human or veterinary patient, in a variety of forms. The form can be specifically adapted to a chosen route of administration, e.g., oral or parenteral administration, by intravenous, intramuscular, inhalation, such as intranasal, or subcutaneous routes.

In some embodiments, the compounds described herein can be formulated for use in treating or preventing a SARS-CoV-2 infection in a human. In some embodiments, the compounds described herein can be formulated with a pharmaceutically acceptable carrier for use in treating or preventing a SARS-CoV-2 infection. In other embodiments, the compounds described herein can be formulated for oral administration, inhalation, or injection for use in treating or preventing a SARS-CoV-2 infection.

The disclosed compounds can be used alone, in combination with one another, or as an adjunct to, or in combination with, other established therapies. In some examples, one disclosed compound is used alone, but in other examples, 2 or more of the disclosed compounds, such as 2, 3, 4, 5, or more of the disclosed compounds, can be used in combination, and can be administered simultaneously or sequentially in any order, and by the same or a different route of administration. In some embodiments, a combination of the disclosed compounds comprises two or more of the 5′End-1, 5′End-2, 5′End-3, TRS-1, TRS-2, and TRS-3 nucleic acid base sequences.

Additionally, or alternatively, the disclosed compound(s) can be used in combination with other therapeutic agents useful for treating and/or preventing SARS-CoV-2 infections. These compounds can be administered simultaneously, sequentially in any order, by the same route of administration, or by a different route.

For nasal administration or administration by inhalation or insufflation, the active compound(s), and/or a pharmaceutically acceptable salt, can be conveniently delivered in the form of an aerosol spray from pressurized packs or a nebulizer with the use of a suitable propellant, for example, dichlorodifluoromethane, trichlorofluoromethane, dichlorotetrafluoroethane, fluorocarbons, carbon dioxide or other suitable gas. In the case of a pressurized aerosol, the dosage unit can be determined by providing a valve to deliver a metered amount. Capsules and cartridges for use in an inhaler or insufflator (for example capsules and cartridges comprised of gelatin) can be formulated containing a powder mix of the compound and a suitable powder base such as lactose or starch. The compound can be dissolved in water or other suitable aqueous solution and aerosolized for inhalation. Alternatively, the compound can be provided as a dry powder suitable for inhalation.

The compounds described herein can be systemically administered in combination with a pharmaceutically acceptable vehicle, such as an inert diluent or an assimilable edible carrier. For oral administration, compounds can be enclosed in hard- or soft-shell gelatin capsules, compressed into tablets, or incorporated directly into the food of a patient's diet. Compounds can also be combined with one or more excipients and used in the form of ingestible tablets, buccal tablets, troches, capsules, elixirs, suspensions, syrups, wafers, and the like. Such compositions and preparations typically contain at least 0.1% of active compound. The percentage of the compositions and preparations can vary and can conveniently be from about 2% to about 60% of the weight of a given unit dosage form. The amount of active compound in such therapeutically useful compositions is such that an effective dosage level can be obtained.

The tablets, troches, pills, capsules, and the like can also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose or aspartame; or a flavoring agent such as peppermint, oil of wintergreen, or cherry flavoring, can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac or sugar and the like. A syrup or elixir can contain the active compound, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the active compound can be incorporated into sustained-release preparations and devices.

The active compound(s) can be administered intravenously or intraperitoneally by infusion or injection. Solutions of the active compound(s) or its salts can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations can contain a preservative to prevent the growth of microorganisms.

Pharmaceutical dosage forms suitable for injection or infusion can include sterile aqueous solutions, dispersions, or sterile powders comprising the active ingredient adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the active compound(s) in the required amount in the appropriate solvent with various of the other ingredients enumerated above, as required, followed by filter sterilization. In the case of sterile powders for the preparation of sterile injectable solutions, methods of preparation can include vacuum drying and freeze drying techniques, which yield a powder of the active ingredient plus any additional desired ingredient present in the previously sterile-filtered solutions.

For any route of administration, the compounds described herein can be used to prepare therapeutic pharmaceutical compositions. In some embodiments, the compound(s) is soluble in water or dilute saline solution, such as an isotonic or less than isotonic saline solution. In other embodiments, the compound(s) can be added to the compositions in the form of a salt or solvate. For example, in cases where compounds are sufficiently basic or acidic to form stable nontoxic acid or base salts, administration of the compounds as salts can be appropriate. Examples of pharmaceutically acceptable salts are organic acid addition salts formed with acids that form a physiological acceptable anion, for example, tosylate, methanesulfonate, acetate, citrate, malonate, tartrate, succinate, benzoate, ascorbate, a-ketoglutarate, and b-glycerophosphate. Suitable inorganic salts can also be formed, including hydrochloride, halide, sulfate, nitrate, bicarbonate, and carbonate salts.

Pharmaceutically acceptable salts can be obtained using procedures known to persons of ordinary skill in the art, for example by reacting a sufficiently basic compound, such as an amine, with a suitable acid to provide a physiologically acceptable ionic compound. Alkali metal (for example, sodium, potassium or lithium) or alkaline earth metal (for example, calcium) salts of carboxylic acids can also be prepared by analogous methods.

B. Dosage

The disclosed compound(s), pharmaceutical compositions and/or combinations thereof will generally be used in an effective amount to treat and/or prevent SARS-CoV-2 infection in a subject, such as a human or non-human animal, particularly a mammal. The disclosed compound(s), or pharmaceutical compositions thereof, can be administered therapeutically to achieve therapeutic benefit or prophylactically to achieve a prophylactic benefit. Therapeutic benefit means amelioration or eradication of a SARS-CoV-2 infection and/or an improvement, such as an easing or ceasing, of one or more symptoms associated with a SARS-CoV-2 infection, such that the subject experiences and/or reports an improvement in feeling or condition, even if the subject is still infected with the SARS-CoV-2 virus. Symptoms of SARS-CoV-2 that can be improved by administering one or more of the disclosed compounds include, but are not limited to, a fever, cough, such as a dry cough, difficulty breathing, shortness of breath, muscle or body aches, pain or pressure in the chest, fatigue, nasal congestion and/or sore throat. Therapeutic benefit also includes halting or slowing the progression of disease caused by SARS-CoV-2, regardless of whether improvement is realized.

In some embodiments, the disclosed compound(s) are formulated to deliver from 0.01 mg/kg to about 30 mg/kg of the compound for use in treating or preventing a SARS-CoV-2 infection.

A person of ordinary skill in the art understands that a preferred dosage of one or more of the disclosed compounds can depend on various factors, including the age, weight, general health, and severity of the condition of the subject being treated. Dosage can also be tailored to the sex of the individual and/or the lung capacity of the individual, when administered by inhalation. Additionally, dosages can be individually tailored for subjects having an underlying condition in addition to SARS-CoV-2, and/or subjects who have additional conditions that affect lung capacity and/or the ability to breath normally. Underlying conditions can include, but are not limited to, blood disorders, such as sickle cell disease or taking blood thinners; chronic kidney or liver disease; conditions that weaken the immune system, such as cancer or cancer treatment, organ or bone marrow transplant, immunosuppressant medications, HIV or AIDS; current or recent pregnancy in the last two weeks; diabetes; inherited metabolic disorders and mitochondrial disorders; heart disease, including coronary artery disease, congenital heart disease, and heart failure; lung disease, including asthma, or COPD; neurological and neurologic and neurodevelopment conditions such as cerebral palsy, epilepsy (seizure disorders), stroke, muscular dystrophy, or spinal cord injury; or a combination thereof. Dosage and frequency of administration of the disclosed compound(s) or pharmaceutical compositions thereof, also will depend on whether the disclosed compound(s) are formulated and/or administered for treatment of a SARS-CoV-2 infection, are formulated and/or administered prophylactically to prevent a SARS-CoV-2 infection, or are formulated for use in the treatment or prevention of a SARS-CoV-2 infection. A person of ordinary skill in the art will be able to determine the optimal dose for a particular individual.

For prophylactic administration, the disclosed compound(s), or pharmaceutical compositions thereof, can be administered to a subject at risk of being infected by the SARS-CoV-2 virus. For example, if a subject works in the medical field with patients suffering from SARS-CoV-2 infections, the disclosed compound(s), or a pharmaceutical composition thereof, can be administered to help prevent the subject from becoming infected. Additionally, or alternatively, the disclosed compound(s), or pharmaceutical compositions thereof, can be administered to a subject having one or more underlying conditions that can make them more at risk of developing serious disease from a SARS-CoV-2 infection, such as one or more of the underlying conditions listed herein.

Effective dosages can be estimated initially from in vitro assays. For example, an initial dosage for use in subjects can be formulated to achieve a circulating blood or serum concentration of active compound that is at or above an IC50 or EC5o of the particular compound as measured in an in vitro assay. Dosages can be calculated to achieve such circulating blood or serum concentrations taking into account the bioavailability of the particular compound. Fingl & Woodbury, “General Principles,” In: Goodman and Gilman's The Pharmaceutical Basis of Therapeutics, Chapter 1, pages 1-46, Pergamon Press, and the references cited therein, provide additional guidance concerning effective dosages.

Initial dosages can also be estimated from in vivo data, such as animal models. For dosage estimation for human administration, suitable animal models can either be animals selected or genetically modified to be susceptible to infection by human strains of SARS-CoV-2, or dosages can be estimated from administration to animals infected with a suitable animal analog of SARS-CoV-2. Persons of ordinary skill in the art can adapt such information to determine dosages suitable for human administration. See e.g., Reagan-Shaw et al., describing a formula for dose translation based on body surface area, the contents of which are incorporated by reference (Reagan-Shaw S, Nihal M, Ahmad N. Dose translation from animal to human studies revisited. FASEB J. 2008 Mar;22(3):659-61. doi: 10.1096/fj.07-9574LSF. Epub 2007 Oct 17. PMID: 17942826).

Dosage amounts of disclosed compound(s) will typically be in the range of from greater than 0 mg/kg/day, such as 0.0001 mg/kg/day or 0.001 mg/kg/day or 0.01 mg/kg/day, up to at least about 100 mg/kg/day. More typically, the dosage (or effective amount) can range from about 0.0025 mg/kg to about 50 mg/kg administered at least once per day, such as from 0.01 mg/kg to about 30 mg/kg, from 0.01 mg/kg to about 20 mg/kg, from 0.01 mg/kg to about 10 mg/kg, or from about 0.05 mg/kg to about 5 mg/kg. The total daily dosage typically ranges from about 0.1 mg/kg to about 100 mg/kg or to about 30 mg/kg per day, such as from 0.5 mg/kg to about 20 mg/kg per day, or from 0.5 mg/kg to about 10 mg/kg per day. Dosage amounts can be higher or lower depending upon, among other factors, the activity of the disclosed compound, its bioavailability, the mode of administration, and various factors discussed above.

In some embodiments, for intranasal administration in humans, a dose can be 1 mg/kg.

Dosage amount and dosage interval can be adjusted for subjects to maintain a therapeutic or prophylactic effect. Dosage amount and dosage interval can also be adjusted based on the compound's use as a medicament. For example, the compound(s) can be administered once per day, multiple times per day, such as 2, 3, 4 or more time per day, once per week, multiple times per week (for example, 2, 3, 4, 5, 6, or 7 times a week, or every other day), one per month, multiple times per month (for example, 2, 3, 4, 5, 6, 7, 8, 9, 10 or more times a month), or once per year, depending upon, amongst other things, the mode of administration, the severity of symptoms with respect to a therapeutic administration, the likelihood of infection with respect to prophylactic administration, and the judgment of the prescribing physician. Persons of ordinary skill in the art will be able to optimize effective local dosages without undue experimentation.

Preferably, the disclosed compound, combinations of disclosed compounds, or pharmaceutical compositions thereof, will provide therapeutic or prophylactic benefit without causing substantial toxicity to a subject. Toxicity of the disclosed compound can be determined using standard pharmaceutical procedures known to persons of ordinary skill in the art. The dose ratio between toxic and therapeutic (or prophylactic) effect is the therapeutic index. Disclosed compounds that exhibit high therapeutic indices are preferred.

C. Additional Therapies

The disclosed compound(s), or pharmaceutical compositions thereof, can be administered alone or in combination with one or more additional therapies. In some embodiments, are described the disclosed compound(s) formulated with one or more additional therapies for use as a medicament. Suitable additional therapies include any therapy that can be administered to treat an underlying condition, to ameliorate one or more symptoms of SARS-CoV-2 infection, and/or to treat or prevent a SARS-CoV-2 infection. In some embodiments, the disclosed compound(s), or pharmaceutical compositions thereof, are administered in combination with, but are not limited to, an antibiotic, anti-inflammatory agent (such as a steroidal anti-inflammatory agent or a nonsteroidal anti-inflammatory agent), analgesic, antiviral, antibody, or a combination thereof. Exemplary analgesics include, but are not limited to, morpholine, hydromorphone, oxycodone, codeine, acetaminophen, hydrocodone, buprenorphine, tramadol, fentanyl, meperidine, pentazocine, or combinations thereof. Exemplary antibiotics include, but are not limited to, penicillins, aminoglycosides, quinolones, cephalosporins, tetracyclines, sulfonamides, macrolides, nitrofurans, or combinations thereof. Exemplary anti-inflammatory agents include, but are not limited to, budesonide, aminosalicylates, cyclooxygenase inhibitors, ibuprofen, naproxen, ketoprofen, or a combination thereof. Exemplary antiviral compounds include, but are not limited to, remdesivir, favilavir, ritonavir, lopinavir, or a combination thereof.

V. Materials and Methods

PPMO synthesis: PPMO were synthesized by covalently conjugating PMO (obtained from Gene Tools, LLC, Philomath, OR) to the cell-penetrating peptide (RXR)4 (where R is arginine and X is 6-aminohexanoic acid) through a noncleavable linker at the 3′ end of each PMO, by methods described herein.

Cells and viruses: Vero E6 cells (ATCC) were propagated in complete growth medium consisting of Dulbecco's modification of Eagle's medium (DMEM) supplemented with 10% heat inactivated fetal bovine serum (FBS) and antibiotics (100 unit/ml penicillin and 100 g/ml streptomycin). All cell culture incubations were carried out at 37° C. in a humidified atmosphere containing 5% CO2. For virus infections, infection media was used, which consisted of DMEM with antibiotics as above, but without serum. SARS-CoV-2 was obtained from CDC. Preparation and quantification of the virus followed methods as previously described by Harcourt, J., et al., Severe Acute Respiratory Syndrome Coronavirus 2 from Patient with 2019 Novel Coronavirus Disease, United States, Emerg. Infect. Dis., 2020. 26(6).

PPMO treatment of virus-infected cell cultures. PPMO were resuspended in sterile PBS. On the day before infection, Vero-E6 cells were plated in 48 well plates at 3×10⁴ cells per well in complete growth medium, resulting in approximately 80% confluence on the day of infection. At 5 hours before infection, the medium was removed and replaced with infection medium containing PPMO. For viral infections, the PPMO-containing medium was aspirated and the cells rinsed twice with infection medium before adding 100 μl of infection medium containing a virus at a multiplicity of infection of 0.01. Following a one-hour infection period, the virus-containing inoculums was aspirated and the cells washed twice with infection medium, after which 300 μl growth medium per well was added. At the indicated time points, all of the media in a well was collected and stored at 4° C. until qPCR or TCID50 analysis, both of which commenced at less than 48 hours after sample collection.

Evaluation of virus quantity by qRT-PCR. Cell supernatants were harvested at indicated time points and viral RNA purified and quantified by using one-step quantitative reverse transcription PCR (qRT-PCR) following methods described by Sheahan, T. P., et al. (Sheahan, T. P., et al. An orally bioavailable broad-spectrum antiviral inhibits SARS-CoV-2 in human airway epithelial cell cultures and multiple coronaviruses in mice, Sci. Transl. Med., 2020).

TCID50 evaluation. Viral supernatants were serially diluted in DMEM and each dilution sample was titrated in triplicate. TCID50/ml values were determined by crystal-violet staining and subsequent scoring of the wells showing cytopathic effect, using the statistical method of Reed and Muench (1938).

### EXAMPLES

### Example 1

### Synthesis of PPMO

The delivery peptide (RAhxRRAhxRRAhxRRAhxRAhxB, R=Arginine, Ahx=6-aminohexanoic acid, B=beta-alanine; SEQ ID NO. 21) and five PMO of sequences listed in Table 1 were purchased from a peptide supplier and Gene Tools LLC (Philomath, Oreg.), respectively. For conjugation of the peptide to the PMO, the PMO was dissolved in dimethylsufoxide (DMSO) at about 100 mg/mL. The peptide solution was made by dissolving peptide powder in DMSO (100mg/mL). The peptide solution (1 eq) was activated by first adding HBTU (1 eq) and followed by adding N,N-diisopropylethylamine (DIEA) (1 eq). Immediately after the addition of DIEA, the peptide solution was mixed and added to the PMO solution at a peptide to PMO reaction ratio of 1.5 to 1. After 2 hours at 45° C., the reaction mixture was diluted with a threefold excess of water. The crude conjugate was purified by strong cation exchange liquid chromatography using a Tricorn Source 15s HPLC column (GE Healthcare, Piscataway, N.J.). Elution of the sample was carried out via a linear NaCl gradient in a 20 mM pH =7 sodium phosphate buffer containing 25% (v:v) acetonitrile. The desired fractions were pooled, desalted by a solid phase extraction method and analyzed by HPLC and mass spectrometry. The product was then quantified and lyophilized.

Evaluation of PPMO targeted against various regions of the 5′ UTR of the SARS-CoV-2 genome.

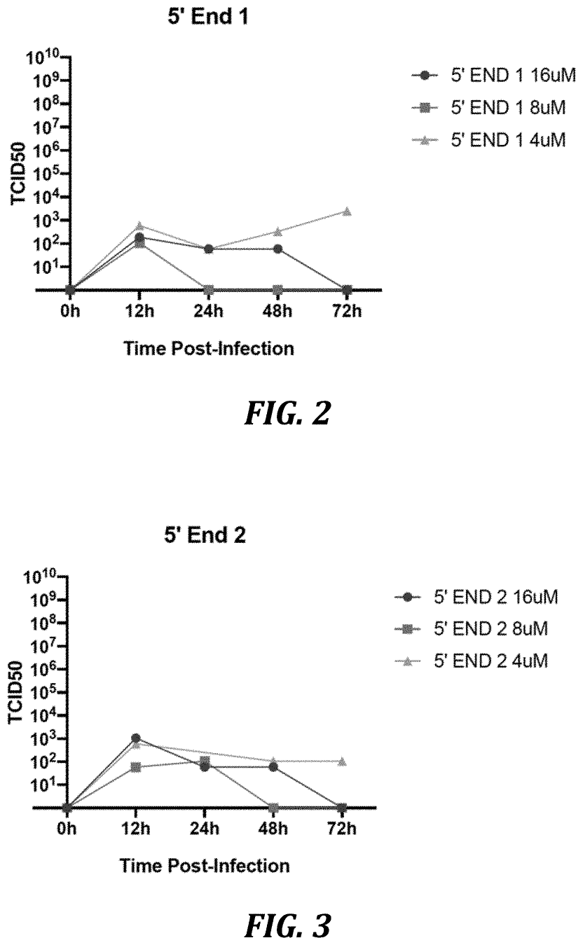

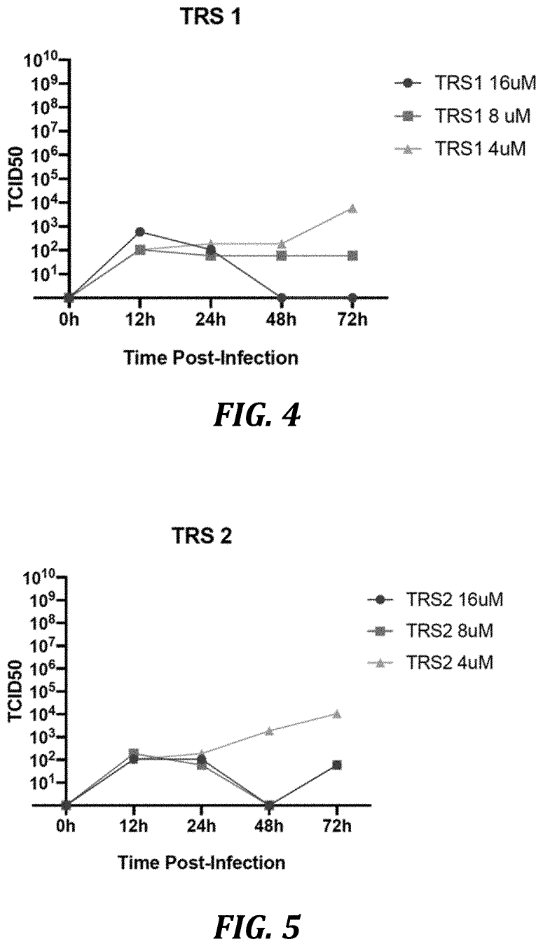

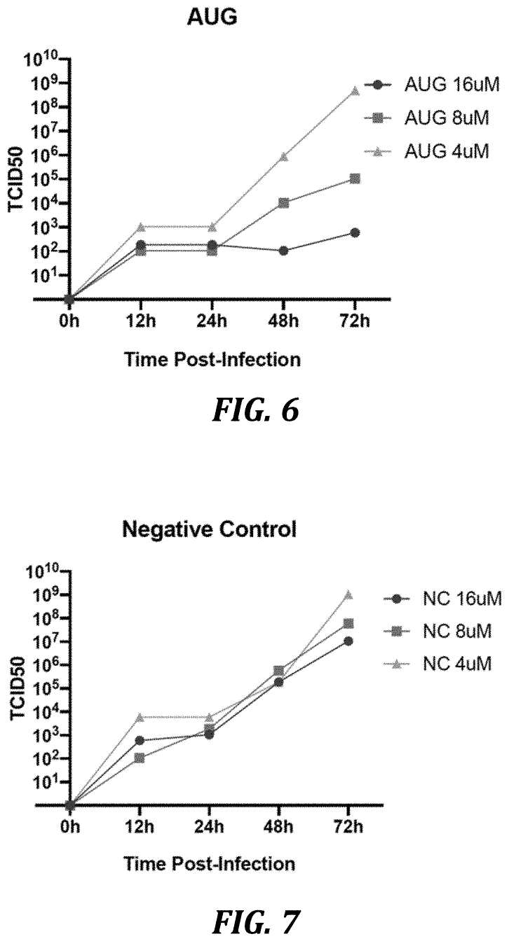

To determine the inhibitory activity of the PPMO on SARS-CoV-2 replication, Vero cells were treated with the five PPMO described in Table 1 at three concentrations: 4, 8, and 16 μM, for 5 hours before infection, then incubated in the absence of PPMO after infection. Cell supernatants were collected at four time-points post-infection: 12, 24, 48, and 72 hours. This test was carried out in 48 well plates, with each set of conditions consisting of a specific PPMO at a single concentration and time-point of supernatant harvest, occupying a single well. Viral titer was evaluated primarily with the use of TCID50 assay, which measures the production of infectious virus. qRT-PCR, which measures the relative number of copies of a segment of viral RNA was also employed in order to have a secondary assay for the level of virus under each set of conditions. Overall, four of the five PPMO which were designed to target SARS-CoV-2 RNA were extremely effective, suppressing viral titers by several orders of magnitude at the 48 and 72 hour time-points (FIGS. 2-6). FIG. 7 provides negative control data, corresponding to a PPMO having a nucleic acid base sequence CCTCTTACCTCAGTTACAATTTATA (SEQ ID NO: 20).

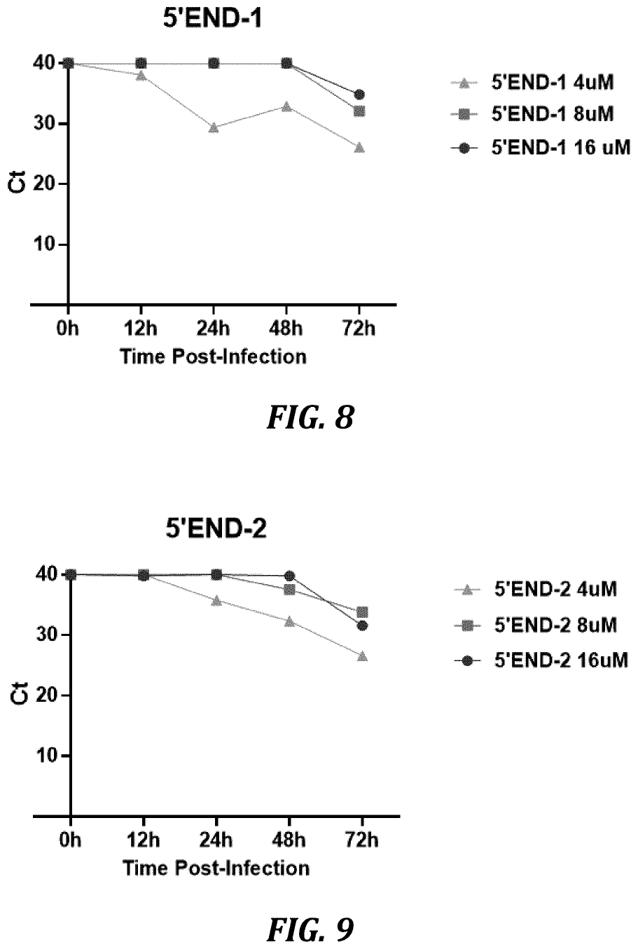

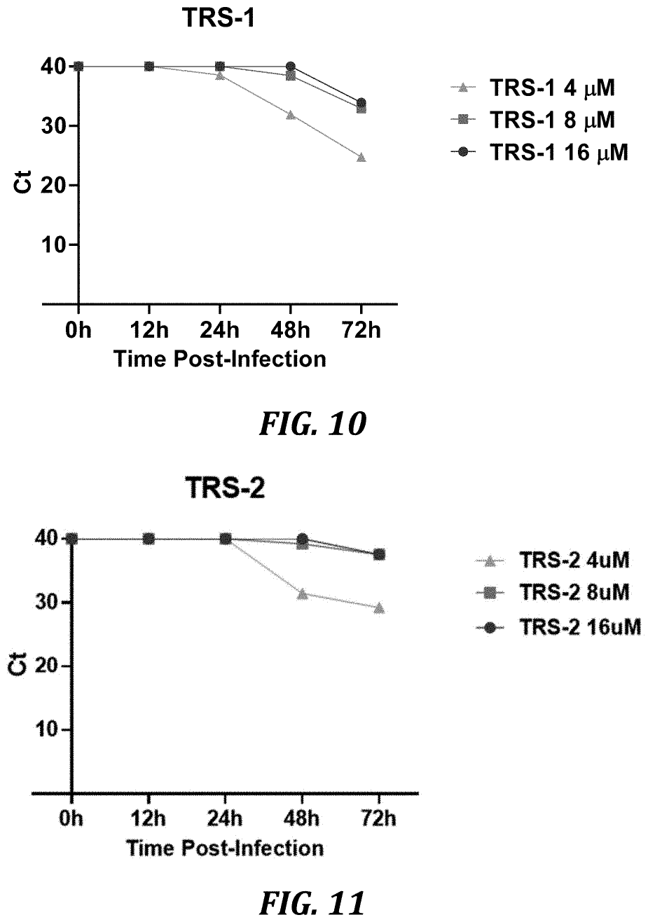

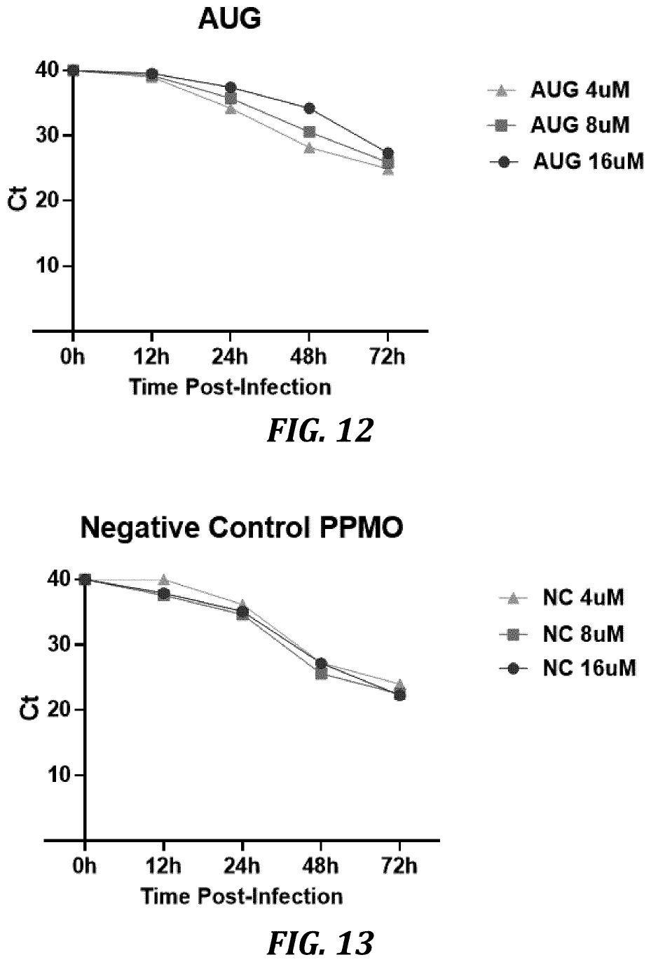

qRT-PCR data obtained from the same experimental samples validates the TCID50 data. qRT-PCR measures the number of amplification cycles (Ct) required to detect a specific segment of viral nucleic acid and provides a measurement of relative quantity of viral genomes present. A rule of thumb is that a 10-cycle difference is equivalent to at least 3 log 10 of viral genomes (i.e., 1000-fold difference). In the data of FIGS. 8-13, the negative control PPMO at all concentrations (and the PBS control sample, not shown) as well as the AUG-PPMO at the lowest concentration used (4 μM), required only around 25 cycles to detect viral nucleic acid, whereas the four most effective PPMO (5′END-1, 5′END-2, TRS-1, TRS-2) when used at concentrations of 8 or 16 μM, required 38-40 cycles up to 48 hours post-infection and 32-38 cycles 72 hours post-infection to detect viral nucleic acid. These data indicate at least a 3 log₁₀ (99.9%) difference in the amount of viral genomes detected between the four most effective PPMO (5′END-1, 5′END-2, TRS-1, TRS-2) when used at concentrations of 8 or 16 μM and the control NC PPMO.

Together, the data identify that the 5′ terminal- and TRS-leader regions of the 5′UTR of SARS-CoV-2 genomic RNA is highly sensitive to PPMO intervention. PPMO-mediated steric blockade of the RNA sequences in these regions results in marked suppression of virus replication. PPMO targeting these regions can therefore be useful inhibitors for treating and/or preventing SARS-CoV-2 infections.

### Example 2

### In Vivo Efficacy

PPMO compounds were evaluated in a mouse model of SARS-CoV-2 infection and disease.

Design:

Mice strain: 129S1;

Date of birth: Feb. 8, 2022;

Virus: SARS-CoV-2 Beta (40 μl of 10⁴ pfu/ml);

Treatment: PPMO (10 mg/kg dose/mouse) TRS-1 (SEQ ID NO: 4) and 5′END-2 (SEQ ID NO: 3);

Treatment time: (1) 18 hours before infection; and (2) 18 hours after infection;

Day of Necropsy: day 3 post-infection;

Other treatment: anesthesia (e.g., ketamine/xylazine) given by intraperitoneal injection during treatment and infection; and

Endpoint: (1) plaque assays and (2) histopathological staining of lung tissue samples taken 3 days post-infection.

For in vivo experiments, mice were infected with SARS-CoV-2 via intranasal inoculation. The mice received the PPMO (TRS-1 (SEQ ID NO: 4) and 5′END-2 (SEQ ID NO: 3) treatments by intranasal administration. The dose level for PPMO was (10 mg/kg dose/mouse). Each experimental group received a first PPMO dose 18 hours before infection, and a second dose 18 hours after infection. On day 3 post-infection, the mice were humanely euthanized and lung tissue samples taken for plaque assays and histopathological staining. Viral titer will be evaluated primarily with the use of TCID50 assay, which measures the production of infectious virus.

Treatment Groups

TABLE 3
treatment groups

| Group (n = 5) | Treatment | Infection |
|---|---|---|
| 1 | Study control | No |
| | (mice received PBS as mock treatment) | |
| 2 | Virus infection control | Yes |
| | (mice received PBS as mock treatment) | |
| 3 | SARS2-TRS-1 | Yes |
| 4 | SARS2-5′END-2 | Yes |
| 5 | NC705 (Negative control PPMO) | Yes |

Plaque Assay

Plaque assays were performed following a standard protocol well-known to those with ordinary skill in the art. See e.g., Mendoza et al., describing a SARS-CoV-2 plaque assay, the contents of which are incorporated by reference (Mendoza E J, Manguiat K, Wood H, Drebot M. Two Detailed Plaque Assay Protocols for the Quantification of Infectious SARS-CoV-2. Curr Protoc Microbiol. 2020 June; 57(1):ecpmc105. doi: 10.1002/cpmc.105. PMID: 32475066; PMCID: PMC7300432).

Results

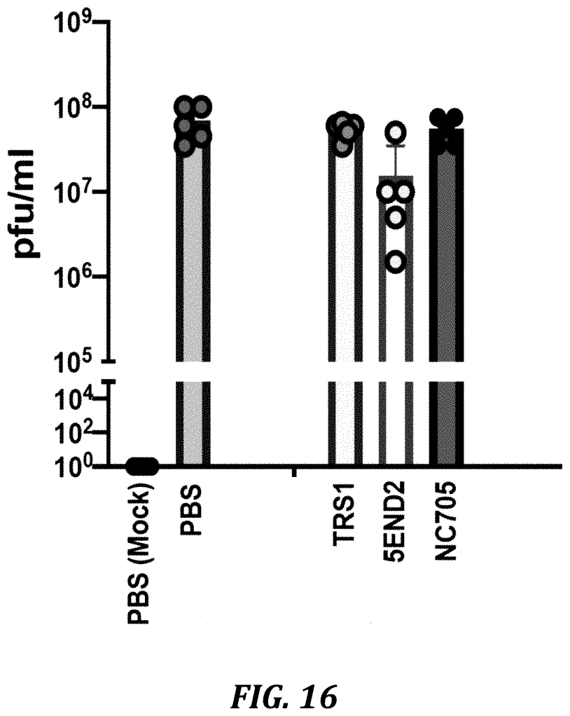

As illustrated in FIG. 16, plaque assay data demonstrate that PPMO 5′END-2 was able to suppress viral titer by approximately 80-90%. In contrast, TRS-1 did not significantly suppress viral titer, as the virus titer in this treatment group was similar to virus infection control (PBS) and the negative control PPMO (NC705).

Anticipated Results in Human

In view of the mouse data, similar results can be anticipated in humans. The PPMO can be administered prophylactically, if exposure to SARS-CoV-2 was suspected. The PPMO can also be administered post-exposure; administration can be as early after infection as possible (e.g., after the first onset of symptoms). The PPMO can be administered at a dose of approximately 1 mg/kg, the PPMO can be administered by intranasal spray on a daily basis without the assistance of a medical professional. As disclosed in this Example, it is expected that the PPMO 5′END-2 can suppress viral titer, similar to what was observed in the mouse treatment group.

Example 3

### PPMO 5′END-3 and TRS-3 Sequence Design

As with other RNA viruses having a positive-sense single-stranded genome (Baltimore Classification, Group IV), the high rate of genetic variant production in coronaviruses is attributed to the large population size, short generation time, and high mutation rate of the viruses. The high mutation rate of coronaviruses is due in large part to the lack of a proof-reading mechanism associated with its RNA polymerase. However, sites within the genome vary in the rate at which they are present in a mutated form compared to their ancestors. In SARS-CoV-2, the 5′UTR contains regions of conserved sequence and RNA-structures as well which have been shown to have critical functions in the processes of translation and synthesis of viral RNA. Included in these regions of conserved sequence is stem-loop 1 (SL1) (located at nucleotides 6-35), which functions in the pre-initiation of translation and also forms a binding site for the viral protein NSP1, a regulator of viral and host translation. Another region of highly conserved RNA in the 5′ UTR contains the transcriptional regulatory sequence leader (TRS-L) (located at nt 70-75), that participates in the formation of the long-range RNA interactions necessary for discontinuous subgenomic mRNA transcription, a process used by all beta-coronaviruses to produce their mRNAs.

To design PPMO targeting SARS-CoV-2, the PPMO sequence design (specifically the sequence of the PMO component) was guided by previous studies using PPMO against various Nidoviruses and other positive-sense single-stranded RNA viruses. Sequence design criteria included: i) targeting regions of the RNA viral genome known to have critical roles in the virus life cycle, ii) targeting specific sites having high sequence conservation across the SARS-CoV-2 virome and iii) targeting regions previously established as being sensitive to PPMO intervention.

As more information about sequence variability and the exact location of the TRS-leader sequence became available, this information was incorporated into new PPMO design. In the time that elapsed since the original PPMO were designed in early 2020, numerous SARS-CoV-2 isolates have been sequenced, and it became apparent that there is considerable sequence variation at nt 1-5 of SARS-CoV-2 across different virus strains. The inventors therefore designed a novel PPMO (5′END-3) that targets nt 6-30 of SARS-CoV-2, in order to obtain a higher degree of target conservation than what was present in 5′END-1 or 5′END-2 PPMO. However, recent analysis showed that 5′END-3 is virtually identical to 5′END-2 in its level of target conservation. See Example 4. This is perhaps unsurprising considering 5′END-2 and 5′END-3 target almost the exact same sequence, differing by only a single nt on the 5′ and 3′ ends.

The inventors also designed a third PPMO to target the TRS-leader region (TRS-3) in the SARS-CoV-2 5′UTR. It is now established that all of the SARS-CoV-2 subgenomic mRNAs likely include the first 75 bases of genomic RNA sequence, but are unlikely to include sequence 3′ from base 75 of the 5′ UTR. The two existing TRS-leader-targeted PPMO target bases 59-82 (TRS-1) and 53-77 (TRS-2). The inventors therefore redesigned a PPMO to target bases 51-75, to improve the likelihood of binding to all subgenomic RNAs, as well as genomic RNA. Table 1 describes all PPMO currently undergoing evaluation.

### Example 4

### Bioinformatic Analysis of Sequence Conservation of PPMO Targets Across the SARS-CoV-2 Virome

The following sequence conservation analysis was performed to determine the relative coverage afforded by the disclosed PPMOs. See Table 1. To do this, the percentage of total SARS-CoV-2 sequences that are perfectly matched (i.e. complementary) with the PPMO was determined, and as well the percentage of SARS-CoV-2 sequences which have either 1, 2, 3, or more mismatches with the PPMO. The inventors' previous work has shown that PPMO have highest efficacy if they have 0 or 1 nt mismatch with their target. The results of the bioinformatics survey demonstrate that 5′END-3 has virtually the same coverage as 5′END-2 (˜92% of SARS-CoV-2 sequences meeting the criteria for inclusion), and that all the TRS sequence targets are very highly conserved (>98% sequences meeting the criteria for inclusion).

TABLE 4
bioinformatics survey

| | Total sequences with |
|---|---|
| | full alignment to the |
| Location | corresponding location |

| | | | |
|---|---|---|---|
| NT 1-24 | mismatches | percent | 3,654 |
| | 0 | 89.33% | |
| | 1 | 6.57% | |
| | 2 | 1.94% | |
| | 3 | 0.68% | |
| | 4 or more | 1.48% | |
| NT 5-29 | mismatches | percent | 13,256 |
| | 0 | 92.32% | |
| | 1 | 6.74% | |
| | 2 | 0.31% | |
| | 3 | 0.15% | |
| | 4 or more | 0.48% | |
| NT 6-30 | mismatches | percent | 14,779 |
| | 0 | 92.75% | |
| | 1 | 6.81% | |
| | 2 | 0.26% | |
| | 3 | 0.08% | |
| | 4 or more | 0.09% | |
| NT 51-75 | mismatches | percent | 142,579 |
| | 0 | 98.58% | |
| | 1 | 0.64% | |
| | 2 | 0.27% | |
| | 3 | 0.02% | |
| | 4 or more | 0.48% | |
| NT 53-77 | mismatches | percent | 143,080 |
| | 0 | 98.27% | |
| | 1 | 0.64% | |
| | 2 | 1.09% | |
| | 3 | 0.01% | |
| | 4 or more | 0.00% | |
| NT 59-82 | mismatches | percent | 310,744 |
| | 0 | 99.59% | |
| | 1 | 0.38% | |
| | 2 | 0.02% | |
| | 3 | 0.01% | |
| | 4 or more | 0.01% | |

### Bioinformatic Methods

Human sequences collected between 15 Jan. 2022 and 14 Jul. 2022 were downloaded from GISAID's EpiCoV database. Only sequences with a length greater than 29,000 bp and with less than 1% Ns were retained. This criteria resulted in a set of 377,137 sequences. “Makeblastdb” was used to build a nucleotide database from these 377,137 sequences, then queried the sequences of interest using “blastn” with the following parameters: word_size: 7; evalue: 6,000,000; penalty: −1; and reward: 2.

TABLE 5
RNA sequence for Severe acute respiratory syndrome coronavirus 2
(SARS-CoV-2) isolate Wuhan-Hu-1, complete genome

```
        1 attaaaggtt tataccttcc caggtaacaa accaaccaac tttcgatctc ttgtagatct
      61 gttctctaaa cgaactttaa aatctgtgtg gctgtcactc ggctgcatgc ttagtgcact
    121 cacgcagtat aattaataac taattactgt cgttgacagg acacgagtaa ctcgtctatc
    181 ttctgcaggc tgcttacggt ttcgtccgtg ttgcagccga tcatcagcac atctaggttt
    241 cgtccgggtg tgaccgaaag gtaagatgga gagccttgtc cctggtttca acgagaaaac
    301 acacgtccaa ctcagtttgc ctgttttaca ggttcgcgac gtgctcgtac gtggctttgg
    361 agactccgtg gaggaggtct tatcagaggc acgtcaacat cttaaagatg gcacttgtgg
    421 cttagtagaa gttgaaaaag gcgttttgcc tcaacttgaa cagccctatg tgttcatcaa
    481 acgttcggat gctcgaactg cacctcatgg tcatgttatg gttgagctgg tagcagaact
    541 cgaaggcatt cagtacggtc gtagtggtga gacacttggt gtccttgtcc ctcatgtggg
    601 cgaaatacca gtggcttacc gcaaggttct tcttcgtaag aacggtaata aaggagctgg
    661 tggccatagt tacggcgccg atctaaagtc atttgactta ggcgacgagc ttggcactga
    721 tccttatgaa gattttcaag aaaactggaa cactaaacat agcagtggtg ttacccgtga
    781 actcatgcgt gagcttaacg gaggggcata cactcgctat gtcgataaca acttctgtgg
    841 ccctgatggc taccctcttg agtgcattaa agaccttcta gcacgtgctg gtaaagcttc
    901 atgcactttg tccgaacaac tggactttat tgacactaag aggggtgtat actgctgccg
    961 tgaacatgag catgaaattg cttggtacac ggaacgttct gaaaagagct atgaattgca
  1021 gacacctttt gaaattaaat tggcaaagaa atttgacacc ttcaatgggg aatgtccaaa
  1081 ttttgtattt cccttaaatt ccataatcaa gactattcaa ccaagggttg aaaagaaaaa
  1141 gcttgatggc tttatgggta gaattcgatc tgtctatcca gttgcgtcac caaatgaatg
  1201 caaccaaatg tgcctttcaa ctctcatgaa gtgtgatcat tgtggtgaaa cttcatggca
  1261 gacgggcgat tttgttaaag ccacttgcga attttgtggc actgagaatt tgactaaaga
  1321 aggtgccact acttgtggtt acttacccca aaatgctgtt gttaaaattt attgtccagc
  1381 atgtcacaat tcagaagtag gacctgagca tagtcttgcc gaataccata atgaatctgg
  1441 cttgaaaacc attcttcgta agggtggtcg cactattgcc tttggaggct gtgtgttctc
  1501 ttatgttggt tgccataaca agtgtgccta ttgggttcca cgtgctagcg ctaacatagg
  1561 ttgtaaccat acaggtgttg ttggagaagg ttccgaaggt cttaatgaca accttcttga
  1621 aatactccaa aaagagaaag tcaacatcaa tattgttggt gactttaaac ttaatgaaga
  1681 gatcgccatt attttggcat ctttttctgc ttccacaagt gcttttgtgg aaactgtgaa
  1741 aggtttggat tataaagcat tcaaacaaat tgttgaatcc tgtggtaatt ttaaagttac
  1801 aaaaggaaaa gctaaaaaag gtgcctggaa tattggtgaa cagaaatcaa tactgagtcc
  1861 tctttatgca tttgcatcag aggctgctcg tgttgtacga tcaattttct cccgcactct
  1921 tgaaactgct caaaattctg tgcgtgtttt acagaaggcc gctataacaa tactagatgg
  1981 aatttcacag tattcactga gactcattga tgctatgatg ttcacatctg atttggctac
  2041 taacaatcta gttgtaatgg cctacattac aggtggtgtt gttcagttga cttcgcagtg
  2101 gctaactaac atctttggca ctgtttatga aaaactcaaa cccgtccttg attggcttga
  2161 agagaagttt aaggaaggtg tagagtttct tagagacggt tgggaaattg ttaaatttat
  2221 ctcaacctgt gcttgtgaaa ttgtcggtgg acaaattgtc acctgtgcaa aggaaattaa
  2281 ggagagtgtt cagacattct ttaagcttgt aaataaattt ttggctttgt gtgctgactc
  2341 tatcattatt ggtggagcta aacttaaagc cttgaattta ggtgaaacat ttgtcacgca
  2401 ctcaaaggga ttgtacagaa agtgtgttaa atccagagaa gaaactggcc tactcatgcc
  2461 tctaaaagcc ccaaaagaaa ttatcttctt agagggagaa acacttccca cagaagtgtt
  2521 aacagaggaa gttgtcttga aaactggtga tttacaacca ttagaacaac ctactagtga
  2581 agctgttgaa gctccattgg ttggtacacc agtttgtatt aacgggctta tgttgctcga
  2641 aatcaaagac acagaaaagt actgtgccct tgcacctaat atgatggtaa caaacaatac
  2701 cttcacactc aaaggcggtg caccaacaaa ggttactttt ggtgatgaca ctgtgataga
  2761 agtgcaaggt tacaagagtg tgaatatcac ttttgaactt gatgaaagga ttgataaagt
  2821 acttaatgag aagtgctctg cctatacagt tgaactcggt acagaagtaa atgagttcgc
  2881 ctgtgttgtg gcagatgctg tcataaaaac tttgcaacca gtatctgaat tacttacacc
  2941 actgggcatt gatttagatg agtggagtat ggctacatac tacttatttg atgagtctgg
  3001 tgagtttaaa ttggcttcac atatgtattg ttctttctac cctccagatg aggatgaaga
  3061 agaaggtgat tgtgaagaag aagagtttga gccatcaact caatatgagt atggtactga
  3121 agatgattac caaggtaaac ctttggaatt tggtgccact tctgctgctc ttcaacctga
  3181 agaagagcaa gaagaagatt ggttagatga tgatagtcaa caaactgttg gtcaacaaga
  3241 cggcagtgag gacaatcaga caactactat tcaaacaatt gttgaggttc aacctcaatt
  3301 agagatggaa cttacaccag ttgttcagac tattgaagtg aatagtttta gtggttattt
  3361 aaaacttact gacaatgtat acattaaaaa tgcagacatt gtggaagaag ctaaaaaggt
  3421 aaaaccaaca gtggttgtta atgcagccaa tgtttacctt aaacatggag gaggtgttgc
  3481 aggagcctta aataaggcta ctaacaatgc catgcaagtt gaatetgatg attacatagc
  3541 tactaatgga ccacttaaag tgggtggtag ttgtgtttta agcggacaca atcttgctaa
  3601 acactgtctt catgttgtcg gcccaaatgt taacaaaggt gaagacattc aacttcttaa
  3661 gagtgcttat gaaaatttta atcagcacga agttctactt gcaccattat tatcagctgg
  3721 tatttttggt gctgacccta tacattcttt aagagtttgt gtagatactg ttcgcacaaa
  3781 tgtctactta gctgtctttg ataaaaatct ctatgacaaa cttgtttcaa gctttttgga
  3841 aatgaagagt gaaaagcaag ttgaacaaaa gatcgctgag attcctaaag aggaagttaa
  3901 gccatttata actgaaagta aaccttcagt tgaacagaga aaacaagatg ataagaaaat
  3961 caaagcttgt gttgaagaag ttacaacaac tctggaagaa actaagttcc tcacagaaaa
  4021 cttgttactt tatattgaca ttaatggcaa tcttcatcca gattctgcca ctcttgttag
  4081 tgacattgac atcactttct taaagaaaga tgctccatat atagtgggtg atgttgttca
  4141 agagggtgtt ttaactgctg tggttatacc tactaaaaag gctggtggca ctactgaaat
  4201 gctagcgaaa gctttgagaa aagtgccaac agacaattat ataaccactt acccgggtca
  4261 gggtttaaat ggttacactg tagaggaggc aaagacagtg cttaaaaagt gtaaaagtgc
  4321 cttttacatt ctaccatcta ttatctctaa tgagaagcaa gaaattcttg gaactgtttc
  4381 ttggaatttg cgagaaatgc ttgcacatgc agaagaaaca cgcaaattaa tgcctgtctg
  4441 tgtggaaact aaagccatag tttcaactat acagcgtaaa tataagggta ttaaaataca
  4501 agagggtgtg gttgattatg gtgctagatt ttacttttac accagtaaaa caactgtagc
  4561 gtcacttatc aacacactta acgatctaaa tgaaactctt gttacaatgc cacttggcta
  4621 tgtaacacat ggcttaaatt tggaagaagc tgctcggtat atgagatctc tcaaagtgcc
  4681 agctacagtt tctgtttctt cacctgatgc tgttacagcg tataatggtt atcttacttc
  4741 ttcttctaaa acacctgaag aacattttat tgaaaccatc tcacttgctg gttcctataa
  4801 agattggtcc tattctggac aatctacaca actaggtata gaatttctta agagaggtga
  4861 taaaagtgta tattacacta gtaatcctac cacattccac ctagatggtg aagttatcac
  4921 ctttgacaat cttaagacac ttctttcttt gagagaagtg aggactatta aggtgtttac
  4981 aacagtagac aacattaacc tccacacgca agttgtggac atgtcaatga catatggaca
  5041 acagtttggt ccaacttatt tggatggagc tgatgttact aaaataaaac ctcataattc
  5101 acatgaaggt aaaacatttt atgttttacc taatgatgac actctacgtg ttgaggcttt
  5161 tgagtactac cacacaactg atcctagttt tctgggtagg tacatgtcag cattaaatca
  5221 cactaaaaag tggaaatacc cacaagttaa tggtttaact tctattaaat gggcagataa
  5281 caactgttat cttgccactg cattgttaac actccaacaa atagagttga agtttaatcc
  5341 acctgctcta caagatgctt attacagagc aagggctggt gaagctgcta acttttgtgc
  5401 acttatctta gcctactgta ataagacagt aggtgagtta ggtgatgtta gagaaacaat
  5461 gagttacttg tttcaacatg ccaatttaga ttcttgcaaa agagtcttga acgtggtgtg
  5521 taaaacttgt ggacaacagc agacaaccct taagggtgta gaagctgtta tgtacatggg
  5581 cacactttct tatgaacaat ttaagaaagg tgttcagata ccttgtacgt gtggtaaaca
  5641 agctacaaaa tatctagtac aacaggagtc accttttgtt atgatgtcag caccacctgc
  5701 tcagtatgaa cttaagcatg gtacatttac ttgtgctagt gagtacactg gtaattacca
  5761 gtgtggtcac tataaacata taacttctaa agaaactttg tattgcatag acggtgcttt
  5821 acttacaaag tcctcagaat acaaaggtcc tattacggat gttttctaca aagaaaacag
  5881 ttacacaaca accataaaac cagttactta taaattggat ggtgttgttt gtacagaaat
  5941 tgaccctaag ttggacaatt attataagaa agacaattct tatttcacag agcaaccaat
  6001 tgatcttgta ccaaaccaac catatccaaa cgcaagcttc gataatttta agtttgtatg
  6061 tgataatatc aaatttgctg atgatttaaa ccagttaact ggttataaga aacctgcttc
  6121 aagagagctt aaagttacat ttttecctga cttaaatggt gatgtggtgg ctattgatta
  6181 taaacactac acaccctctt ttaagaaagg agctaaattg ttacataaac ctattgtttg
  6241 gcatgttaac aatgcaacta ataaagccac gtataaacca aatacctggt gtatacgttg
  6301 tctttggagc acaaaaccag ttgaaacatc aaattcgttt gatgtactga agtcagagga
  6361 cgcgcaggga atggataatc ttgcctgcga agatctaaaa ccagtctctg aagaagtagt
  6421 ggaaaatcct accatacaga aagacgttct tgagtgtaat gtgaaaacta ccgaagttgt
  6481 aggagacatt atacttaaac cagcaaataa tagtttaaaa attacagaag aggttggcca
  6541 cacagatcta atggctgctt atgtagacaa ttctagtctt actattaaga aacctaatga
  6601 attatctaga gtattaggtt tgaaaaccct tgctactcat ggtttagctg ctgttaatag
  6661 tgtcccttgg gatactatag ctaattatgc taagcctttt cttaacaaag ttgttagtac
  6721 aactactaac atagttacac ggtgtttaaa ccgtgtttgt actaattata tgccttattt
  6781 ctttacttta ttgctacaat tgtgtacttt tactagaagt acaaattcta gaattaaagc
  6841 atctatgccg actactatag caaagaatac tgttaagagt gtcggtaaat tttgtctaga
  6901 ggcttcattt aattatttga agtcacctaa tttttctaaa ctgataaata ttataatttg
  6961 gtttttacta ttaagtgttt gcctaggttc tttaatctac tcaaccgctg ctttaggtgt
  7021 tttaatgtct aatttaggca tgccttctta ctgtactggt tacagagaag gctatttgaa
  7081 ctctactaat gtcactattg caacctactg tactggttct ataccttgta gtgtttgtct
  7141 tagtggttta gattctttag acacctatcc ttctttagaa actatacaaa ttaccatttc
  7201 atcttttaaa tgggatttaa ctgcttttgg cttagttgca gagtggtttt tggcatatat
  7261 tcttttcact aggtttttct atgtacttgg attggctgca atcatgcaat tgtttttcag
  7321 ctattttgca gtacatttta ttagtaattc ttggcttatg tggttaataa ttaatcttgt
  7381 acaaatggcc ccgatttcag ctatggttag aatgtacatc ttctttgcat cattttatta
  7441 tgtatggaaa agttatgtgc atgttgtaga cggttgtaat tcatcaactt gtatgatgtg
  7501 ttacaaacgt aatagagcaa caagagtcga atgtacaact attgttaatg gtgttagaag
  7561 gtccttttat gtctatgcta atggaggtaa aggcttttgc aaactacaca attggaattg
  7621 tgttaattgt gatacattct gtgctggtag tacatttatt agtgatgaag ttgcgagaga
  7681 cttgtcacta cagtttaaaa gaccaataaa tcctactgac cagtcttctt acatcgttga
  7741 tagtgttaca gtgaagaatg gttccatcca tctttacttt gataaagctg gtcaaaagac
  7801 ttatgaaaga cattctctct ctcattttgt taacttagac aacctgagag ctaataacac
  7861 taaaggttca ttgcctatta atgttatagt ttttgatggt aaatcaaaat gtgaagaatc
  7921 atctgcaaaa tcagcgtctg tttactacag tcagcttatg tgtcaaccta tactgttact
  7981 agatcaggca ttagtgtctg atgttggtga tagtgcggaa gttgcagtta aaatgtttga
  8041 tgcttacgtt aatacgtttt catcaacttt taacgtacca atggaaaaac tcaaaacact
  8101 agttgcaact gcagaagctg aacttgcaaa gaatgtgtcc ttagacaatg tcttatctac
  8161 ttttatttca gcagctcggc aagggtttgt tgattcagat gtagaaacta aagatgttgt
  8221 tgaatgtctt aaattgtcac atcaatctga catagaagtt actggcgata gttgtaataa
  8281 ctatatgctc acctataaca aagttgaaaa catgacaccc cgtgaccttg gtgcttgtat
  8341 tgactgtagt gcgcgtcata ttaatgcgca ggtagcaaaa agtcacaaca ttgctttgat
  8401 atggaacgtt aaagatttca tgtcattgtc tgaacaacta cgaaaacaaa tacgtagtgc
  8461 tgctaaaaag aataacttac cttttaagtt gacatgtgca actactagac aagttgttaa
  8521 tgttgtaaca acaaagatag cacttaaggg tggtaaaatt gttaataatt ggttgaagca
  8581 gttaattaaa gttacacttg tgttcctttt tgttgctgct attttctatt taataacacc
  8641 tgttcatgtc atgtctaaac atactgactt ttcaagtgaa atcataggat acaaggctat
  8701 tgatggtggt gtcactcgtg acatagcatc tacagatact tgttttgcta acaaacatgc
  8761 tgattttgac acatggttta gccagcgtgg tggtagttat actaatgaca aagcttgccc
  8821 attgattgct gcagtcataa caagagaagt gggttttgtc gtgcctggtt tgcctggcac
  8881 gatattacgc acaactaatg gtgacttttt gcatttctta cctagagttt ttagtgcagt
  8941 tggtaacatc tgttacacac catcaaaact tatagagtac actgactttg caacatcagc
  9001 ttgtgttttg gctgctgaat gtacaatttt taaagatgct tctggtaagc cagtaccata
  9061 ttgttatgat accaatgtac tagaaggttc tgttgcttat gaaagtttac gccctgacac
  9121 acgttatgtg ctcatggatg gctctattat tcaatttcct aacacctacc ttgaaggttc
  9181 tgttagagtg gtaacaactt ttgattctga gtactgtagg cacggcactt gtgaaagatc
  9241 agaagctggt gtttgtgtat ctactagtgg tagatgggta cttaacaatg attattacag
  9301 atctttacca ggagttttct gtggtgtaga tgctgtaaat ttacttacta atatgtttac
  9361 accactaatt caacctattg gtgctttgga catatcagca tctatagtag ctggtggtat
  9421 tgtagctatc gtagtaacat gccttgccta ctattttatg aggtttagaa gagcttttgg
  9481 tgaatacagt catgtagttg cctttaatac tttactattc cttatgtcat tcactgtact
  9541 ctgtttaaca ccagtttact cattcttacc tggtgtttat tctgttattt acttgtactt
  9601 gacattttat cttactaatg atgtttcttt tttagcacat attcagtgga tggttatgtt
  9661 cacaccttta gtacctttct ggataacaat tgcttatatc atttgtattt ccacaaagca
  9721 tttctattgg ttctttagta attacctaaa gagacgtgta gtctttaatg gtgtttcctt
  9781 tagtactttt gaagaagctg cgctgtgcac ctttttgtta aataaagaaa tgtatctaaa
  9841 gttgcgtagt gatgtgctat tacctcttac gcaatataat agatacttag ctctttataa
  9901 taagtacaag tattttagtg gagcaatgga tacaactagc tacagagaag ctgcttgttg
  9961 tcatctcgca aaggctctca atgacttcag taactcaggt tctgatgttc tttaccaacc
10021 accacaaacc tctatcacct cagctgtttt gcagagtggt tttagaaaaa tggcattccc
10081 atctggtaaa gttgagggtt gtatggtaca agtaacttgt ggtacaacta cacttaacgg
10141 tctttggctt gatgacgtag tttactgtcc aagacatgtg atctgcacct ctgaagacat
10201 gcttaaccct aattatgaag atttactcat tcgtaagtct aatcataatt tcttggtaca
10261 ggctggtaat gttcaactca gggttattgg acattctatg caaaattgtg tacttaagct
10321 taaggttgat acagccaatc ctaagacacc taagtataag tttgttcgca ttcaaccagg
10381 acagactttt tcagtgttag cttgttacaa tggttcacca tctggtgttt accaatgtgc
10441 tatgaggccc aatttcacta ttaagggttc attccttaat ggttcatgtg gtagtgttgg
10501 ttttaacata gattatgact gtgtctcttt ttgttacatg caccatatgg aattaccaac
10561 tggagttcat gctggcacag acttagaagg taacttttat ggaccttttg ttgacaggca
10621 aacagcacaa gcagctggta cggacacaac tattacagtt aatgttttag cttggttgta
10681 cgctgctgtt ataaatggag acaggtggtt tctcaatcga tttaccacaa ctcttaatga
10741 ctttaacctt gtggctatga agtacaatta tgaacctcta acacaagacc atgttgacat
10801 actaggacct ctttctgctc aaactggaat tgccgtttta gatatgtgtg cttcattaaa
10861 agaattactg caaaatggta tgaatggacg taccatattg ggtagtgctt tattagaaga
10921 tgaatttaca ccttttgatg ttgttagaca atgctcaggt gttactttcc aaagtgcagt
10981 gaaaagaaca atcaagggta cacaccactg gttgttactc acaattttga cttcactttt
11041 agttttagtc cagagtactc aatggtcttt gttctttttt ttgtatgaaa atgccttttt
11101 accttttgct atgggtatta ttgctatgtc tgcttttgca atgatgtttg tcaaacataa
11161 gcatgcattt ctctgtttgt ttttgttacc ttctcttgcc actgtagctt attttaatat
11221 ggtctatatg cctgctagtt gggtgatgcg tattatgaca tggttggata tggttgatac
11281 tagtttgtct ggttttaagc taaaagactg tgttatgtat gcatcagctg tagtgttact
11341 aatccttatg acagcaagaa ctgtgtatga tgatggtgct aggagagtgt ggacacttat
11401 gaatgtcttg acactcgttt ataaagttta ttatggtaat gctttagatc aagccatttc
11461 catgtgggct cttataatct ctgttacttc taactactca ggtgtagtta caactgtcat
11521 gtttttggcc agaggtattg tttttatgtg tgttgagtat tgccctattt tcttcataac
11581 tggtaataca cttcagtgta taatgctagt ttattgtttc ttaggctatt tttgtacttg
11641 ttactttggc ctcttttgtt tactcaaccg ctactttaga ctgactcttg gtgtttatga
11701 ttacttagtt tctacacagg agtttagata tatgaattca cagggactac tcccacccaa
11761 gaatagcata gatgccttca aactcaacat taaattgttg ggtgttggtg gcaaaccttg
11821 tatcaaagta gccactgtac agtctaaaat gtcagatgta aagtgcacat cagtagtctt
11881 actctcagtt ttgcaacaac tcagagtaga atcatcatct aaattgtggg ctcaatgtgt
11941 ccagttacac aatgacattc tcttagctaa agatactact gaagcctttg aaaaaatggt
12001 ttcactactt tctgttttgc tttccatgca gggtgctgta gacataaaca agctttgtga
12061 agaaatgctg gacaacaggg caaccttaca agctatagcc tcagagttta gttcccttcc
12121 atcatatgca gcttttgcta ctgctcaaga agcttatgag caggctgttg ctaatggtga
12181 ttctgaagtt gttcttaaaa agttgaagaa gtctttgaat gtggctaaat ctgaatttga
12241 ccgtgatgca gccatgcaac gtaagttgga aaagatggct gatcaagcta tgacccaaat
12301 gtataaacag gctagatctg aggacaagag ggcaaaagtt actagtgcta tgcagacaat
12361 gcttttcact atgcttagaa agttggataa tgatgcactc aacaacatta tcaacaatgc
12421 aagagatggt tgtgttccct tgaacataat acctcttaca acagcagcca aactaatggt
12481 tgtcatacca gactataaca catataaaaa tacgtgtgat ggtacaacat ttacttatgc
12541 atcagcattg tgggaaatcc aacaggttgt agatgcagat agtaaaattg ttcaacttag
12601 tgaaattagt atggacaatt cacctaattt agcatggcct cttattgtaa cagctttaag
12661 ggccaattct gctgtcaaat tacagaataa tgagcttagt cctgttgcac tacgacagat
12721 gtcttgtgct gccggtacta cacaaactgc ttgcactgat gacaatgcgt tagcttacta
12781 caacacaaca aagggaggta ggtttgtact tgcactgtta tccgatttac aggatttgaa
12841 atgggctaga ttecctaaga gtgatggaac tggtactatc tatacagaac tggaaccacc
12901 ttgtaggttt gttacagaca cacctaaagg tcctaaagtg aagtatttat actttattaa
12961 aggattaaac aacctaaata gaggtatggt acttggtagt ttagctgcca cagtacgtct
13021 acaagctggt aatgcaacag aagtgcctgc caattcaact gtattatctt tctgtgcttt
13081 tgctgtagat gctgctaaag cttacaaaga ttatctagct agtgggggac aaccaatcac
13141 taattgtgtt aagatgttgt gtacacacac tggtactggt caggcaataa cagttacacc
13201 ggaagccaat atggatcaag aatcctttgg tggtgcatcg tgttgtctgt actgccgttg
13261 ccacatagat catccaaatc ctaaaggatt ttgtgactta aaaggtaagt atgtacaaat
13321 acctacaact tgtgctaatg accctgtggg ttttacactt aaaaacacag tctgtaccgt
13381 ctgcggtatg tggaaaggtt atggctgtag ttgtgatcaa ctccgcgaac ccatgcttca
13441 gtcagctgat gcacaatcgt ttttaaacgg gtttgcggtg taagtgcagc ccgtcttaca
13501 ccgtgcggca caggcactag tactgatgtc gtatacaggg cttttgacat ctacaatgat
13561 aaagtagctg gttttgctaa attcctaaaa actaattgtt gtcgcttcca agaaaaggac
13621 gaagatgaca atttaattga ttcttacttt gtagttaaga gacacacttt ctctaactac
13681 caacatgaag aaacaattta taatttactt aaggattgtc cagctgttgc taaacatgac
13741 ttctttaagt ttagaataga cggtgacatg gtaccacata tatcacgtca acgtcttact
13801 aaatacacaa tggcagacct cgtctatgct ttaaggcatt ttgatgaagg taattgtgac
13861 acattaaaag aaatacttgt cacatacaat tgttgtgatg atgattattt caataaaaag
13921 gactggtatg attttgtaga aaacccagat atattacgcg tatacgccaa cttaggtgaa
13981 cgtgtacgcc aagctttgtt aaaaacagta caattctgtg atgccatgcg aaatgctggt
14041 attgttggtg tactgacatt agataatcaa gatctcaatg gtaactggta tgatttcggt
14101 gatttcatac aaaccacgcc aggtagtgga gttcctgttg tagattctta ttattcattg
14161 ttaatgccta tattaacctt gaccagggct ttaactgcag agtcacatgt tgacactgac
14221 ttaacaaagc cttacattaa gtgggatttg ttaaaatatg acttcacgga agagaggtta
14281 aaactctttg accgttattt taaatattgg gatcagacat accacccaaa ttgtgttaac
14341 tgtttggatg acagatgcat tctgcattgt gcaaacttta atgttttatt ctctacagtg
14401 tteccaccta caagttttgg accactagtg agaaaaatat ttgttgatgg tgttccattt
14461 gtagtttcaa ctggatacca cttcagagag ctaggtgttg tacataatca ggatgtaaac
14521 ttacatagct ctagacttag ttttaaggaa ttacttgtgt atgctgctga ccctgctatg
14581 cacgctgctt ctggtaatct attactagat aaacgcacta cgtgcttttc agtagctgca
14641 cttactaaca atgttgcttt tcaaactgtc aaacccggta attttaacaa agacttctat
14701 gactttgctg tgtctaaggg tttctttaag gaaggaagtt ctgttgaatt aaaacacttc
14761 ttctttgctc aggatggtaa tgctgctatc agcgattatg actactatcg ttataatcta
14821 ccaacaatgt gtgatatcag acaactacta tttgtagttg aagttgttga taagtacttt
14881 gattgttacg atggtggctg tattaatgct aaccaagtca tcgtcaacaa cctagacaaa
14941 tcagctggtt ttccatttaa taaatggggt aaggctagac tttattatga ttcaatgagt
15001 tatgaggatc aagatgcact tttcgcatat acaaaacgta atgtcatccc tactataact
15061 caaatgaatc ttaagtatgc cattagtgca aagaatagag ctegcaccgt agctggtgtc
15121 tctatctgta gtactatgac caatagacag tttcatcaaa aattattgaa atcaatagcc
15181 gccactagag gagctactgt agtaattgga acaagcaaat tctatggtgg ttggcacaac
15241 atgttaaaaa ctgtttatag tgatgtagaa aaccctcacc ttatgggttg ggattatcct
15301 aaatgtgata gagccatgcc taacatgctt agaattatgg cctcacttgt tcttgctcgc
15361 aaacatacaa cgtgttgtag cttgtcacac cgtttctata gattagctaa tgagtgtgct
15421 caagtattga gtgaaatggt catgtgtggc ggttcactat atgttaaacc aggtggaacc
15481 tcatcaggag atgccacaac tgcttatgct aatagtgttt ttaacatttg tcaagctgtc
15541 acggccaatg ttaatgcact tttatctact gatggtaaca aaattgccga taagtatgtc
15601 cgcaatttac aacacagact ttatgagtgt ctctatagaa atagagatgt tgacacagac
15661 tttgtgaatg agttttacgc atatttgcgt aaacatttct caatgatgat actctctgac
15721 gatgctgttg tgtgtttcaa tagcacttat gcatctcaag gtctagtggc tagcataaag
15781 aactttaagt cagttcttta ttatcaaaac aatgttttta tgtctgaagc aaaatgttgg
15841 actgagactg accttactaa aggacctcat gaattttgct ctcaacatac aatgctagtt
15901 aaacagggtg atgattatgt gtaccttcct tacccagatc catcaagaat cctaggggcc
15961 ggctgttttg tagatgatat cgtaaaaaca gatggtacac ttatgattga acggttcgtg
16021 tctttagcta tagatgctta cccacttact aaacatccta atcaggagta tgctgatgtc
16081 tttcatttgt acttacaata cataagaaag ctacatgatg agttaacagg acacatgtta
16141 gacatgtatt ctgttatgct tactaatgat aacacttcaa ggtattggga acctgagttt
16201 tatgaggcta tgtacacacc gcatacagtc ttacaggctg ttggggcttg tgttctttgc
16261 aattcacaga cttcattaag atgtggtgct tgcatacgta gaccattctt atgttgtaaa
16321 tgctgttacg accatgtcat atcaacatca cataaattag tcttgtctgt taatccgtat
16381 gtttgcaatg ctccaggttg tgatgtcaca gatgtgactc aactttactt aggaggtatg
16441 agctattatt gtaaatcaca taaaccaccc attagttttc cattgtgtgc taatggacaa
16501 gtttttggtt tatataaaaa tacatgtgtt ggtagcgata atgttactga ctttaatgca
16561 attgcaacat gtgactggac aaatgctggt gattacattt tagctaacac ctgtactgaa
16621 agactcaagc tttttgcagc agaaacgctc aaagctactg aggagacatt taaactgtct
16681 tatggtattg ctactgtacg tgaagtgctg tctgacagag aattacatct ttcatgggaa
16741 gttggtaaac ctagaccacc acttaaccga aattatgtct ttactggtta tcgtgtaact
16801 aaaaacagta aagtacaaat aggagagtac acctttgaaa aaggtgacta tggtgatgct
16861 gttgtttacc gaggtacaac aacttacaaa ttaaatgttg gtgattattt tgtgctgaca
16921 tcacatacag taatgccatt aagtgcacct acactagtgc cacaagagca ctatgttaga
16981 attactggct tatacccaac actcaatatc tcagatgagt tttctagcaa tgttgcaaat
17041 tatcaaaagg ttggtatgca aaagtattct acactccagg gaccacctgg tactggtaag
17101 agtcattttg ctattggcct agctctctac tacccttctg ctcgcatagt gtatacagct
17161 tgctctcatg ccgctgttga tgcactatgt gagaaggcat taaaatattt gcctatagat
17221 aaatgtagta gaattatacc tgcacgtgct cgtgtagagt gttttgataa attcaaagtg
17281 aattcaacat tagaacagta tgtcttttgt actgtaaatg cattgcctga gacgacagca
17341 gatatagttg tctttgatga aatttcaatg gccacaaatt atgatttgag tgttgtcaat
17401 gccagattac gtgctaagca ctatgtgtac attggcgacc ctgctcaatt acctgcacca
17461 cgcacattgc taactaaggg cacactagaa ccagaatatt tcaattcagt gtgtagactt
17521 atgaaaacta taggtccaga catgttcctc ggaacttgtc ggcgttgtcc tgctgaaatt
17581 gttgacactg tgagtgcttt ggtttatgat aataagctta aagcacataa agacaaatca
17641 gctcaatgct ttaaaatgtt ttataagggt gttatcacgc atgatgtttc atctgcaatt
17701 aacaggccac aaataggcgt ggtaagagaa ttecttacac gtaaccctgc ttggagaaaa
17761 gctgtcttta tttcacctta taattcacag aatgctgtag cctcaaagat tttgggacta
17821 ccaactcaaa ctgttgattc atcacagggc tcagaatatg actatgtcat attcactcaa
17881 accactgaaa cagctcactc ttgtaatgta aacagattta atgttgctat taccagagca
17941 aaagtaggca tactttgcat aatgtctgat agagaccttt atgacaagtt gcaatttaca
18001 agtcttgaaa ttccacgtag gaatgtggca actttacaag ctgaaaatgt aacaggactc
18061 tttaaagatt gtagtaaggt aatcactggg ttacatccta cacaggcacc tacacacctc
18121 agtgttgaca ctaaattcaa aactgaaggt ttatgtgttg acatacctgg catacctaag
18181 gacatgacct atagaagact catctctatg atgggtttta aaatgaatta tcaagttaat
18241 ggttacccta acatgtttat cacccgcgaa gaagctataa gacatgtacg tgcatggatt
18301 ggcttcgatg tcgaggggtg tcatgctact agagaagctg ttggtaccaa tttaccttta
18361 cagctaggtt tttctacagg tgttaaccta gttgctgtac ctacaggtta tgttgataca
18421 cctaataata cagatttttc cagagttagt gctaaaccac cgcctggaga tcaatttaaa
18481 cacctcatac cacttatgta caaaggactt ccttggaatg tagtgcgtat aaagattgta
18541 caaatgttaa gtgacacact taaaaatctc tctgacagag tcgtatttgt cttatgggca
18601 catggctttg agttgacatc tatgaagtat tttgtgaaaa taggacctga gegcacctgt
18661 tgtctatgtg atagacgtgc cacatgcttt tccactgett cagacactta tgcctgttgg
18721 catcattcta ttggatttga ttacgtctat aatccgttta tgattgatgt tcaacaatgg
18781 ggttttacag gtaacctaca aagcaaccat gatctgtatt gtcaagtcca tggtaatgca
18841 catgtagcta gttgtgatgc aatcatgact aggtgtctag ctgtccacga gtgctttgtt
18901 aagcgtgttg actggactat tgaatatcct ataattggtg atgaactgaa gattaatgcg
18961 gcttgtagaa aggttcaaca catggttgtt aaagctgcat tattagcaga caaattccca
19021 gttcttcacg acattggtaa ccctaaagct attaagtgtg tacctcaagc tgatgtagaa
19081 tggaagttct atgatgcaca gccttgtagt gacaaagctt ataaaataga agaattattc
19141 tattcttatg ccacacattc tgacaaattc acagatggtg tatgectatt ttggaattgc
19201 aatgtcgata gatatcctgc taattccatt gtttgtagat ttgacactag agtgctatct
19261 aaccttaact tgcctggttg tgatggtggc agtttgtatg taaataaaca tgcattccac
19321 acaccagctt ttgataaaag tgcttttgtt aatttaaaac aattaccatt tttctattac
19381 tctgacagtc catgtgagtc tcatggaaaa caagtagtgt cagatataga ttatgtacca
19441 ctaaagtctg ctacgtgtat aacacgttgc aatttaggtg gtgctgtctg tagacatcat
19501 gctaatgagt acagattgta tctcgatgct tataacatga tgatctcagc tggctttagc
19561 ttgtgggttt acaaacaatt tgatacttat aacctctgga acacttttac aagacttcag
19621 agtttagaaa atgtggcttt taatgttgta aataagggac actttgatgg acaacagggt
19681 gaagtaccag tttctatcat taataacact gtttacacaa aagttgatgg tgttgatgta
19741 gaattgtttg aaaataaaac aacattacct gttaatgtag catttgagct ttgggctaag
19801 cgcaacatta aaccagtacc agaggtgaaa atactcaata atttgggtgt ggacattgct
19861 gctaatactg tgatctggga ctacaaaaga gatgctccag cacatatatc tactattggt
19921 gtttgttcta tgactgacat agccaagaaa ccaactgaaa cgatttgtgc accactcact
19981 gtcttttttg atggtagagt tgatggtcaa gtagacttat ttagaaatgc ccgtaatggt
20041 gttcttatta cagaaggtag tgttaaaggt ttacaaccat ctgtaggtcc caaacaagct
20101 agtcttaatg gagtcacatt aattggagaa gccgtaaaaa cacagttcaa ttattataag
20161 aaagttgatg gtgttgtcca acaattacct gaaacttact ttactcagag tagaaattta
20221 caagaattta aacccaggag tcaaatggaa attgatttct tagaattagc tatggatgaa
20281 ttcattgaac ggtataaatt agaaggctat gccttcgaac atatcgttta tggagatttt
20341 agtcatagtc agttaggtgg tttacatcta ctgattggac tagctaaacg ttttaaggaa
20401 tcaccttttg aattagaaga ttttattcct atggacagta cagttaaaaa ctatttcata
20461 acagatgcgc aaacaggttc atctaagtgt gtgtgttctg ttattgattt attacttgat
20521 gattttgttg aaataataaa atcccaagat ttatctgtag tttctaaggt tgtcaaagtg
20581 actattgact atacagaaat ttcatttatg ctttggtgta aagatggcca tgtagaaaca
20641 ttttacccaa aattacaatc tagtcaagcg tggcaaccgg gtgttgctat gcctaatctt
20701 tacaaaatgc aaagaatgct attagaaaag tgtgaccttc aaaattatgg tgatagtgca
20761 acattaccta aaggcataat gatgaatgtc gcaaaatata ctcaactgtg tcaatattta
20821 aacacattaa cattagctgt accctataat atgagagtta tacattttgg tgctggttct
20881 gataaaggag ttgcaccagg tacagctgtt ttaagacagt ggttgcctac gggtacgctg
20941 cttgtcgatt cagatcttaa tgactttgtc tctgatgcag attcaacttt gattggtgat
21001 tgtgcaactg tacatacagc taataaatgg gatctcatta ttagtgatat gtacgaccct
21061 aagactaaaa atgttacaaa agaaaatgac tctaaagagg gttttttcac ttacatttgt
21121 gggtttatac aacaaaagct agctcttgga ggttccgtgg ctataaagat aacagaacat
21181 tcttggaatg ctgatcttta taagctcatg ggacacttcg catggtggac agcctttgtt
21241 actaatgtga atgcgtcatc atctgaagca tttttaattg gatgtaatta tcttggcaaa
21301 ccacgcgaac aaatagatgg ttatgtcatg catgcaaatt acatattttg gaggaataca
21361 aatccaattc agttgtcttc ctattcttta tttgacatga gtaaatttcc ccttaaatta
21421 aggggtactg ctgttatgtc tttaaaagaa ggtcaaatca atgatatgat tttatctctt
21481 cttagtaaag gtagacttat aattagagaa aacaacagag ttgttatttc tagtgatgtt
21541 cttgttaaca actaaacgaa caatgtttgt ttttcttgtt ttattgccac tagtctctag
21601 tcagtgtgtt aatcttacaa ccagaactca attaccccct gcatacacta attctttcac
21661 acgtggtgtt tattaccctg acaaagtttt cagatcctca gttttacatt caactcagga
21721 cttgttctta cctttctttt ccaatgttac ttggttccat gctatacatg tctctgggac
21781 caatggtact aagaggtttg ataaccctgt cctaccattt aatgatggtg tttattttgc
21841 ttccactgag aagtctaaca taataagagg ctggattttt ggtactactt tagattcgaa
21901 gacccagtcc ctacttattg ttaataacgc tactaatgtt gttattaaag tctgtgaatt
21961 tcaattttgt aatgatccat ttttgggtgt ttattaccac aaaaacaaca aaagttggat
22021 ggaaagtgag ttcagagttt attctagtgc gaataattgc acttttgaat atgtctctca
22081 gccttttctt atggaccttg aaggaaaaca gggtaatttc aaaaatctta gggaatttgt
22141 gtttaagaat attgatggtt attttaaaat atattctaag cacacgccta ttaatttagt
22201 gcgtgatctc cctcagggtt tttcggcttt agaaccattg gtagatttgc caataggtat
22261 taacatcact aggtttcaaa ctttacttgc tttacataga agttatttga ctcctggtga
22321 ttcttcttca ggttggacag ctggtgctgc agcttattat gtgggttatc ttcaacctag
22381 gacttttcta ttaaaatata atgaaaatgg aaccattaca gatgctgtag actgtgcact
22441 tgaccctctc tcagaaacaa agtgtacgtt gaaatccttc actgtagaaa aaggaatcta
22501 tcaaacttct aactttagag tccaaccaac agaatctatt gttagatttc ctaatattac
22561 aaacttgtgc ccttttggtg aagtttttaa cgccaccaga tttgcatctg tttatgcttg
22621 gaacaggaag agaatcagca actgtgttgc tgattattct gtcctatata attccgcatc
22681 attttccact tttaagtgtt atggagtgtc tcctactaaa ttaaatgatc tctgctttac
22741 taatgtctat gcagattcat ttgtaattag aggtgatgaa gtcagacaaa tcgctccagg
22801 gcaaactgga aagattgctg attataatta taaattacca gatgatttta caggctgcgt
22861 tatagcttgg aattctaaca atcttgattc taaggttggt ggtaattata attacctgta
22921 tagattgttt aggaagtcta atctcaaacc ttttgagaga gatatttcaa ctgaaatcta
22981 tcaggccggt agcacacctt gtaatggtgt tgaaggtttt aattgttact ttcctttaca
23041 atcatatggt ttccaaccca ctaatggtgt tggttaccaa ccatacagag tagtagtact
23101 ttcttttgaa cttctacatg caccagcaac tgtttgtgga cctaaaaagt ctactaattt
23161 ggttaaaaac aaatgtgtca atttcaactt caatggttta acaggcacag gtgttcttac
23221 tgagtctaac aaaaagtttc tgcctttcca acaatttggc agagacattg ctgacactac
23281 tgatgctgtc cgtgatccac agacacttga gattcttgac attacaccat gttcttttgg
23341 tggtgtcagt gttataacac caggaacaaa tacttctaac caggttgctg ttctttatca
23401 ggatgttaac tgcacagaag tccctgttgc tattcatgca gatcaactta ctcctacttg
23461 gcgtgtttat tctacaggtt ctaatgtttt tcaaacacgt gcaggctgtt taataggggc
23521 tgaacatgtc aacaactcat atgagtgtga catacccatt ggtgcaggta tatgcgctag
23581 ttatcagact cagactaatt ctcctcggcg ggcacgtagt gtagctagtc aatccatcat
23641 tgcctacact atgtcacttg gtgcagaaaa ttcagttgct tactctaata actctattgc
23701 catacccaca aattttacta ttagtgttac cacagaaatt ctaccagtgt ctatgaccaa
23761 gacatcagta gattgtacaa tgtacatttg tggtgattca actgaatgca gcaatctttt
23821 gttgcaatat ggcagttttt gtacacaatt aaaccgtgct ttaactggaa tagctgttga
23881 acaagacaaa aacacccaag aagtttttgc acaagtcaaa caaatttaca aaacaccacc
23941 aattaaagat tttggtggtt ttaatttttc acaaatatta ccagatccat caaaaccaag
24001 caagaggtca tttattgaag atctactttt caacaaagtg acacttgcag atgctggctt
24061 catcaaacaa tatggtgatt gccttggtga tattgctgct agagacctca tttgtgcaca
24121 aaagtttaac ggccttactg ttttgccacc tttgctcaca gatgaaatga ttgctcaata
24181 cacttctgca ctgttagcgg gtacaatcac ttctggttgg acctttggtg caggtgctgc
24241 attacaaata ccatttgcta tgcaaatggc ttataggttt aatggtattg gagttacaca
24301 gaatgttctc tatgagaacc aaaaattgat tgccaaccaa tttaatagtg ctattggcaa
24361 aattcaagac tcactttctt ccacagcaag tgcacttgga aaacttcaag atgtggtcaa
24421 ccaaaatgca caagctttaa acacgcttgt taaacaactt agctccaatt ttggtgcaat
24481 ttcaagtgtt ttaaatgata tcctttcacg tcttgacaaa gttgaggctg aagtgcaaat
24541 tgataggttg atcacaggca gacttcaaag tttgcagaca tatgtgactc aacaattaat
24601 tagagctgca gaaatcagag cttctgctaa tcttgctgct actaaaatgt cagagtgtgt
24661 acttggacaa tcaaaaagag ttgatttttg tggaaagggc tatcatctta tgtccttccc
24721 tcagtcagca cctcatggtg tagtcttctt gcatgtgact tatgtccctg cacaagaaaa
24781 gaacttcaca actgctcctg ccatttgtca tgatggaaaa gcacactttc ctcgtgaagg
24841 tgtctttgtt tcaaatggca cacactggtt tgtaacacaa aggaattttt atgaaccaca
24901 aatcattact acagacaaca catttgtgtc tggtaactgt gatgttgtaa taggaattgt
24961 caacaacaca gtttatgatc ctttgcaacc tgaattagac tcattcaagg aggagttaga
25021 taaatatttt aagaatcata catcaccaga tgttgattta ggtgacatct ctggcattaa
25081 tgcttcagtt gtaaacattc aaaaagaaat tgaccgcctc aatgaggttg ccaagaattt
25141 aaatgaatct ctcatcgatc tccaagaact tggaaagtat gagcagtata taaaatggcc
25201 atggtacatt tggctaggtt ttatagctgg cttgattgcc atagtaatgg tgacaattat
25261 gctttgctgt atgaccagtt gctgtagttg tctcaagggc tgttgttctt gtggatcctg
25321 ctgcaaattt gatgaagacg actctgagcc agtgctcaaa ggagtcaaat tacattacac
25381 ataaacgaac ttatggattt gtttatgaga atcttcacaa ttggaactgt aactttgaag
25441 caaggtgaaa tcaaggatgc tactccttca gattttgttc gcgctactgc aacgataccg
25501 atacaagcct cactcccttt cggatggctt attgttggcg ttgcacttct tgctgttttt
25561 cagagcgctt ccaaaatcat aaccctcaaa aagagatggc aactagcact ctccaagggt
25621 gttcactttg tttgcaactt gctgttgttg tttgtaacag tttactcaca ccttttgctc
25681 gttgctgctg gccttgaagc cccttttctc tatctttatg ctttagtcta cttcttgcag
25741 agtataaact ttgtaagaat aataatgagg ctttggcttt gctggaaatg ccgttccaaa
25801 aacccattac tttatgatgc caactatttt ctttgctggc atactaattg ttacgactat
25861 tgtatacctt acaatagtgt aacttcttca attgtcatta cttcaggtga tggcacaaca
25921 agtcctattt ctgaacatga ctaccagatt ggtggttata ctgaaaaatg ggaatctgga
25981 gtaaaagact gtgttgtatt acacagttac ttcacttcag actattacca gctgtactca
26041 actcaattga gtacagacac tggtgttgaa catgttacct tcttcatcta caataaaatt
26101 gttgatgagc ctgaagaaca tgtccaaatt cacacaatcg acggttcatc cggagttgtt
26161 aatccagtaa tggaaccaat ttatgatgaa ccgacgacga ctactagcgt gcctttgtaa
26221 gcacaagctg atgagtacga acttatgtac tcattcgttt cggaagagac aggtacgtta
26281 atagttaata gegtacttct ttttcttgct ttcgtggtat tcttgctagt tacactagcc
26341 atccttactg cgcttcgatt gtgtgcgtac tgctgcaata ttgttaacgt gagtcttgta
26401 aaaccttctt tttacgttta ctctcgtgtt aaaaatctga attcttctag agttcctgat
26461 cttctggtct aaacgaacta aatattatat tagtttttct gtttggaact ttaattttag
26521 ccatggcaga ttccaacggt actattaccg ttgaagagct taaaaagctc cttgaacaat
26581 ggaacctagt aataggtttc ctattcctta catggatttg tcttctacaa tttgcctatg
26641 ccaacaggaa taggtttttg tatataatta agttaatttt cctctggctg ttatggccag
26701 taactttagc ttgttttgtg cttgctgctg tttacagaat aaattggatc accggtggaa
26761 ttgctatcgc aatggcttgt cttgtaggct tgatgtggct cagctacttc attgcttctt
26821 tcagactgtt tgcgcgtacg cgttccatgt ggtcattcaa tccagaaact aacattcttc
26881 tcaacgtgcc actccatggc actattctga ccagaccgct tctagaaagt gaactcgtaa
26941 tcggagctgt gatccttcgt ggacatcttc gtattgctgg acaccatcta ggacgctgtg
27001 acatcaagga cctgcctaaa gaaatcactg ttgctacatc acgaacgctt tcttattaca
27061 aattgggagc ttcgcagcgt gtagcaggtg actcaggttt tgctgcatac agtcgctaca
27121 ggattggcaa ctataaatta aacacagacc attccagtag cagtgacaat attgctttgc
27181 ttgtacagta agtgacaaca gatgtttcat ctcgttgact ttcaggttac tatagcagag
27241 atattactaa ttattatgag gacttttaaa gtttccattt ggaatcttga ttacatcata
27301 aacctcataa ttaaaaattt atctaagtca ctaactgaga ataaatattc tcaattagat
27361 gaagagcaac caatggagat tgattaaacg aacatgaaaa ttattctttt cttggcactg
27421 ataacactcg ctacttgtga gctttatcac taccaagagt gtgttagagg tacaacagta
27481 cttttaaaag aaccttgctc ttctggaaca tacgagggca attcaccatt tcatcctcta
27541 gctgataaca aatttgcact gacttgcttt agcactcaat ttgcttttgc ttgtcctgac
27601 ggcgtaaaac acgtctatca gttacgtgcc agatcagttt cacctaaact gttcatcaga
27661 caagaggaag ttcaagaact ttactctcca atttttctta ttgttgcggc aatagtgttt
27721 ataacacttt gcttcacact caaaagaaag acagaatgat tgaactttca ttaattgact
27781 tctatttgtg ctttttagcc tttctgctat tccttgtttt aattatgctt attatctttt
27841 ggttctcact tgaactgcaa gatcataatg aaacttgtca cgcctaaacg aacatgaaat
27901 ttcttgtttt cttaggaatc atcacaactg tagctgcatt tcaccaagaa tgtagtttac
27961 agtcatgtac tcaacatcaa ccatatgtag ttgatgaccc gtgtcctatt cacttctatt
28021 ctaaatggta tattagagta ggagctagaa aatcagcacc tttaattgaa ttgtgegtgg
28081 atgaggctgg ttctaaatca cccattcagt acatcgatat cggtaattat acagtttcct
28141 gtttaccttt tacaattaat tgccaggaac ctaaattggg tagtcttgta gtgcgttgtt
28201 cgttctatga agacttttta gagtatcatg acgttcgtgt tgttttagat ttcatctaaa
28261 cgaacaaact aaaatgtctg ataatggacc ccaaaatcag cgaaatgcac cccgcattac
28321 gtttggtgga ccctcagatt caactggcag taaccagaat ggagaacgca gtggggcgcg
28381 atcaaaacaa cgtcggcccc aaggtttacc caataatact gcgtcttggt tcaccgctct
28441 cactcaacat ggcaaggaag accttaaatt ccctcgagga caaggcgttc caattaacac
28501 caatagcagt ccagatgacc aaattggcta ctaccgaaga gctaccagac gaattcgtgg
28561 tggtgacggt aaaatgaaag atctcagtcc aagatggtat ttctactacc taggaactgg
28621 gccagaagct ggacttccct atggtgctaa caaagacggc atcatatggg ttgcaactga
28681 gggagccttg aatacaccaa aagatcacat tggcacccgc aatcctgcta acaatgctgc
28741 aatcgtgcta caacttcctc aaggaacaac attgccaaaa ggcttctacg cagaagggag
28801 cagaggcggc agtcaagcct cttctcgttc ctcatcacgt agtcgcaaca gttcaagaaa
28861 ttcaactcca ggcagcagta ggggaacttc tcctgctaga atggctggca atggcggtga
28921 tgctgctctt gctttgctgc tgcttgacag attgaaccag cttgagagca aaatgtctgg
28981 taaaggccaa caacaacaag gccaaactgt cactaagaaa tctgctgctg aggcttctaa
29041 gaagcctcgg caaaaacgta ctgccactaa agcatacaat gtaacacaag ctttcggcag
29101 acgtggtcca gaacaaaccc aaggaaattt tggggaccag gaactaatca gacaaggaac
29161 tgattacaaa cattggccgc aaattgcaca atttgccccc agcgcttcag cgttcttcgg
29221 aatgtcgcgc attggcatgg aagtcacacc ttcgggaacg tggttgacct acacaggtgc
29281 catcaaattg gatgacaaag atccaaattt caaagatcaa gtcattttgc tgaataagca
29341 tattgacgca tacaaaacat tcccaccaac agagcctaaa aaggacaaaa agaagaaggc
29401 tgatgaaact caagccttac cgcagagaca gaagaaacag caaactgtga ctcttcttcc
29461 tgctgcagat ttggatgatt tctccaaaca attgcaacaa tccatgagca gtgctgactc
29521 aactcaggcc taaactcatg cagaccacac aaggcagatg ggctatataa acgttttcgc
29581 ttttccgttt acgatatata gtctactctt gtgcagaatg aattctcgta actacatagc
29641 acaagtagat gtagttaact ttaatctcac atagcaatct ttaatcagtg tgtaacatta
29701 gggaggactt gaaagagcca ccacattttc accgaggcca cgcggagtac gatcgagtgt
29761 acagtgaaca atgctaggga gagctgccta tatggaagag ccctaatgtg taaaattaat
29821 tttagtagtg ctatccccat gtgattttaa tagcttctta ggagaatgac aaaaaaaaaa
29881 aaaaaaaaaa aaaaaaaaaa aaa
```

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

The embodiments of the invention in which an exclusive property or privilege is claimed are defined as follows:

1. A compound comprising:
an oligomer that comprises nucleic acid base sequence antisense to at least a portion of an RNA sequence of SARS-CoV-2, and a backbone comprising moieties that sterically block DNA and/or RNA cleavage.

2. The compound according to claim 1, further comprising a peptide.

3. The compound according to claim 1, wherein the nucleic acid base sequence is antisense to at least a portion of nucleotides 1-285 of the SARS-CoV-2 genomic RNA.

4. The compound according to claim 3, wherein the nucleic acid base sequence is antisense to at least a portion of nucleotides 1-50 of the SARS-CoV-2 genomic RNA.

5. The compound according to claim 1, wherein the SARS-CoV-2 genomic RNA has a sequence with at least 80% sequence identity to the sequence as set forth in SEQ ID NO: 1.

6. The compound according to claim 1, wherein the oligomer comprises a nucleic acid base sequence selected from SEQ ID NOs: 2-19, 22, and 23 or a nucleic acid base sequence having at least 90% sequence identity to one or more of SEQ ID NOs: 2-19, 22, and 23.

7. The compound according to claim 1, wherein the oligomer comprises a nucleic acid base sequence selected from SEQ ID NOs: 2-5, 22, and 23.

8. The compound according to claim 1, wherein the oligomer comprises a nucleic acid base sequence selected from SEQ ID NO: 2, SEQ ID NO: 3, or SEQ ID NO:

22.

9. The compound according to claim 1, wherein the oligomer backbone comprises phosphorodiamidate morpholino (PMO), methylphosphonate, 2′-O-methyl RNA (2′-)Me), 2′-O-methyl phosphorothioate (2′-OMePS), 2′-O-methoxyethyl RNA (2′-MOE), 2′-O-methoxyethyl phosphorothioate (2′-MOE-PS), peptide nucleic acid (PNA), tricycle-DNA (tcDNA), locked nucleic acid (LNA), or a combination thereof.

10. The compound according to claim 1, wherein the oligomer backbone comprises a structure selected from

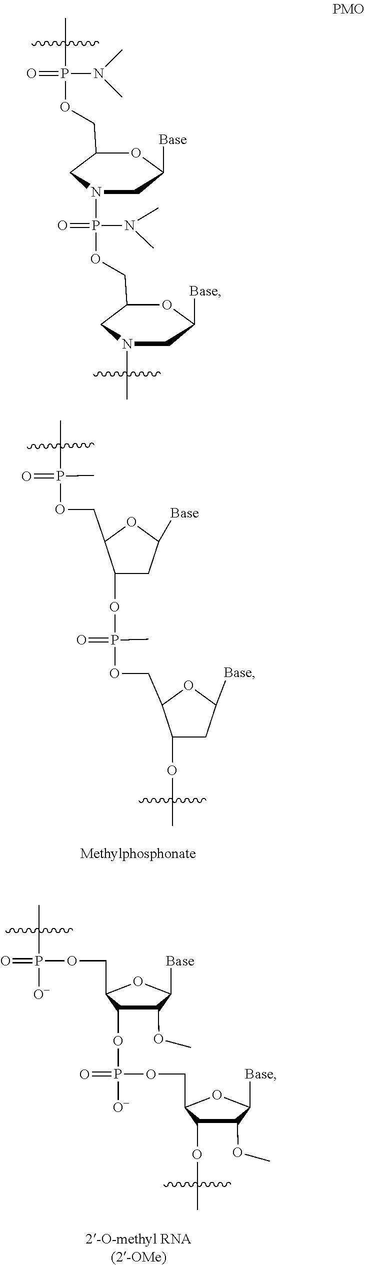

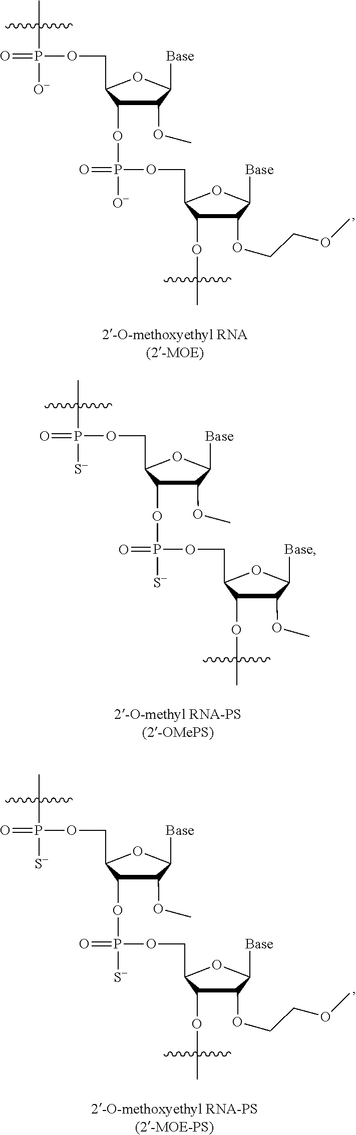

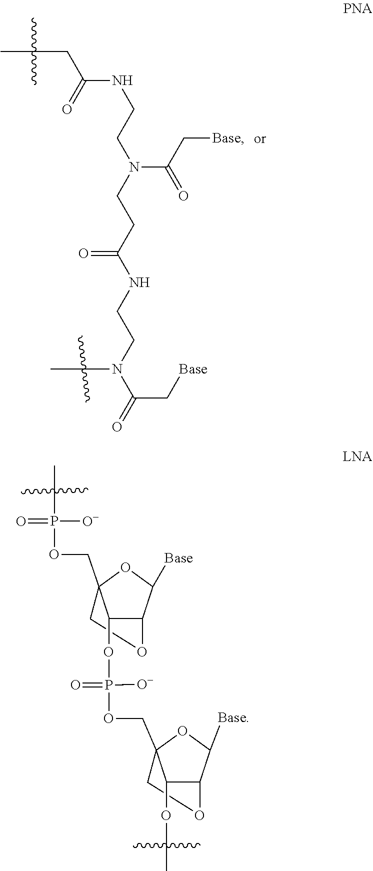

11. The compound according to claim 2, wherein the peptide has a peptide length of from 2 to 60 amino acids.

12. The compound according to claim 2, wherein the peptide comprises one or more amino acids selected from glycine, valine, alanine, leucine, isoleucine, methionine, phenylalanine, tryptophan, tyrosine, serine, threonine, asparagine, glutamine, arginine, histidine, lysine, aspartic acid, glutamic acid, cysteine, proline, beta-alanine, selenocysteine, pyrrolysine, 7-aminoheptanoic acid, 6-amino hexanoic acid, 5-aminopentanoic acid, 4-aminobutanoic acid, homoarginine, or amino acids containing a poly(oxyethylene) group.

13. The compound according to claim 2, wherein the peptide comprises a sequence as set forth in SEQ ID NO: 21, or wherein the peptide has a sequence with at least 90% sequence identity to the sequence as set forth in SEQ ID NO: 21.

14. The compound according to claim 2, wherein the peptide is attached at the 3′ end of the oligomer, wherein the peptide is attached directly to the oligomer backbone or indirectly to the oligomer backbone through a linker.

15. The compound according to claim 2, wherein the peptide is attached at the 5′ end of the oligomer, wherein the peptide is attached directly to the oligomer backbone or indirectly to the oligomer backbone through a linker.

16. The compound according to claim 2, wherein the compound has a structure according to Formula 1 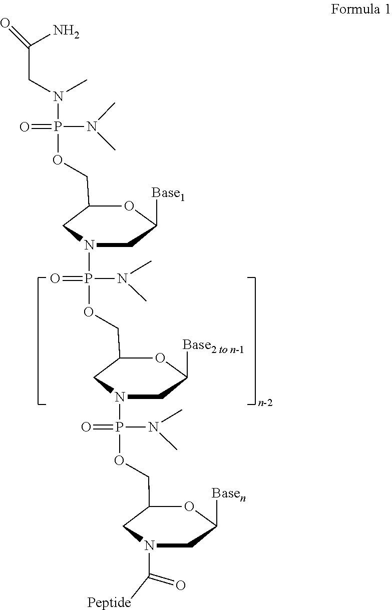
wherein:
n is from 2 to 50;
each base independently is selected from adenine, guanine, cytosine, thymine or uracil; and
peptide is a peptide comprising from 2 amino acid to 60 amino acids.

17. The compound according to claim 16, wherein the compound has a structure according to Formula 2 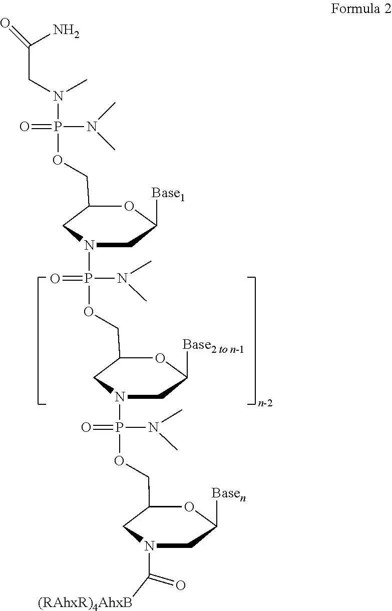
wherein R is Arginine, Ahx is 6-aminohexanoic acid, and B is beta-alanine.

18. The compound according to claim 16, wherein Base₁ to Baseₙ is SEQ ID NO: 2, SEQ ID NO: 3, or SEQ ID NO: 22.

19. A method of treating or preventing a SARS-CoV-2 infection, comprising administering to a subject a compound according to claim 2.

20. A method for treating or preventing a SARS-CoV-2 infection in a human subject, comprising administering to the subject an effective amount of a compound having a structure 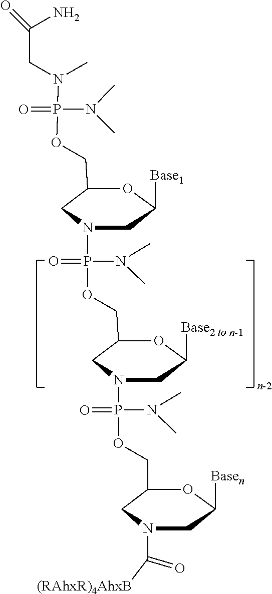
or a pharmaceutically acceptable salt thereof, wherein:
n is from 20 to 30;
each Base independently is selected from adenine, guanine, cytosine or thymine;
R is Arginine;
Ahx is 6-aminohexanoic acid; and
B is beta-alanine.
